(19) United States
(12) Patent Application Publication
(10) Pub. No.: US 2009/0117146 A1
(43) Pub. Date: May 7, 2009

(54) SYSTEM AND METHOD FOR PROMOTING HAIR GROWTH AND IMPROVING HAIR AND SCALP HEALTH

(75) Inventors: Mohammed Alal Khan, Newfoundland (CA); David Callahan, Newfoundland (CA)

Correspondence Address: MAYER & WILLIAMS PC, 251 NORTH AVENUE WEST, 2ND FLOOR, WESTFIELD, NJ, 07090, US

(73) Assignee: Island Laboratories, Inc., St. George's, Newfoundland (CA)

(21) Appl. No.: 11/914,743

(22) PCT Filed: May 16, 2006
(86) PCT No.: PCT/CA06/00765

§ 371 Date: Dec. 1, 2008

Related U.S. Application Data
(60) Provisional application No. 60/681,147, filed on May 16, 2005.

(51) Int. Cl. A61K 36/03; A61K 8/97; A61Q 7/00; A61K 36/00; A61K 36/02; A61K 36/185; A61P 1/00; A61P 17/14

(52) U.S. Cl. 424/195.17; 424/725

(57) ABSTRACT

A system is provided for promoting hair growth comprising one or more extracts from a steroidal alkaloid-containing plant selected from a Veratrum plant, a Buxus plant, a Holarrhena plant, a Solarium plant and a Rauwolfia plant. The system can further comprise an extract from a Pilocarpus plant and a seaweed extract. The system can also be used in reducing hair loss, enhancing or restoring hair colour, increasing the thickness of hair, improving the genera appearance of hair, and/or reducing or eliminating dandruff. Methods of promoting the growth of hair are also provided.

### FIELD OF THE INVENTION

The present invention relates to the field of hair and scalp treatment, and in particular to plant extracts for promoting hair growth.

### BACKGROUND OF THE INVENTION

Alopecia, or hair loss, is an ongoing problem afflicting mankind and animals. Men, woman and children can all suffer from alopecia, which can be the result of one, or a combination of, a number of factors including genetic factors, hormonal factors, surgery, trauma and stress. The universality of the occurrence of alopecia has led to continuing efforts throughout history to discover compositions for stimulating hair growth and preventing hair loss.

Hair is basically composed of a tough and insoluble protein, keratin. Each hair comprises a cylindrical shaft and a root, and is contained in a follicle, which is a flask-like depression in the skin. The average human head, for example, has about 100,000 hair follicles spread across the scalp. The bottom of the follicle contains a finger-like projection termed the papilla, which consists of connective tissue from which hair grows, and through which blood vessels supply the cells with nourishment. The shaft is the part that extends outwards from the skin surface, whilst the root has been described as the buried part of the hair. The base of the root expands into the hair bulb, which rests upon the papilla. Cells from which the hair is produced grow in the bulb of the follicle; they are extruded in the form of fibres as the cells proliferate in the follicle. “Hair growth” refers to the formation and elongation of the hair fibre by the dividing cells. Hair growth is not constant, however, because follicles are cycling through stages of growth, rest and regression. In most mammals, the cycle of hair growth is seasonal and is related to daylight length and hormonal activity, resulting in regular periods of shedding, or moulting. By contrast, human hair follicles generally cycle independently of one another, with each follicle apparently having its own internal clock. Various studies have shown that a range of substances, such as growth factors, hormones, and drugs, can modulate the cycle by increasing the population of stem cells and/or affecting the supply of nutrients to the hair.

While men typically suffer pattern baldness with receding hairlines and bald spots on the crown of the bead, women typically experience generalised thinning of the hair over the entire top of the head. Androgenetic alopccia (or “male pattern baldness”) occurs when the pilar cycle become accelerated or disturbed. In other words, alopecia occurs when the growth phases are shortened and the hairs proceed to the telogen phase earlier, shedding in large numbers. The successive growth cycles lead to increasingly thinner and increasingly shorter hairs, converting gradually to an unpigmented down.

Various factors are believed to contribute to the occurrence of alopecia in an individual including genetics, nutrition, hormone levels (including the levels of thyroid hormones, as well as the steroid hormones testosterone and aldosterone and their precursors), and levels of certain growth factors.

For example, several studies have shown that hair follicles are sensitive to androgens. Testosterone is the principal circulating androgen in humans and is converted to dihydrotestosterone in a reaction catalyzed by the enzyme 5-alpha reductase. The effect of androgens on scalp hair loss may be mediated through changes in intracellular concentrations of cyclic AMP (cAMP). The effect of various sex hormones on the activity of adenyl cyclase in the follicles of scalp hair has been tested indicating that dihydrotestosterone inhibits adenyl cyclase activity, but that testosterone does not. It has been suggested that high dihydrotestosterone levels in hair follicles can initiate baldness by inhibiting adenyl cyclase.

The sonic hedgehog pathway may also play an important role in male and female pattern baldness. In the skin, sonic hedgehog (Shh) is required for hair follicle morphogenesis during embryogenesis and for regulating follicular growth and cycling in the adult. Recent studies indicate that a topically applied hedgehog (Hh)-agonist can modulate follicular cycling in adult mouse skin. The Hh-agonist stimulated the transition form the resting (telogen) to the growth (anagen) stage of the hair cycle in adult mouse skin, suggesting that topical application of Hh-agonist could be effective in treating conditions of decreased proliferation and aberrant follicular cycling in the scalp including androgenetic alopecia (Paladini et al., Journal of Investigative Dermatology. 2005; Volume 125 Page 638).

The FDA has estimated that 300,000 hair growth remedies have been marketed in the United States. One well-known compound currently in clinical use for treating alopecia is 2,4-diamino-6-piperidinopyrimidine 3-oxide (minoxidil or Rogaine). Interest in the hair growing properties of minoxidil surfaced in 1979 with the advent of Loniten tablets, which were approved by the US Food and Drug Administration for the reduction of blood pressure. Approximately 80% of the patients taking Loniten tablets started growing excessive hair on the face, shoulders and trunk and patients with androgenetic alopecia were observed to grow new hair on their heads. Various compositions for the treatment of baldness comprising minoxidil have been described including compositions comprising minoxidil in combination with saw palmetto extract and nettle root extract (see, U.S. Pat. No. 6,596,266 and U.S. Patent Application 20020028257).

A number of more natural remedies for alopecia based solely on herbs and plant extracts have been proposed. For example, compositions for promoting hair growth or reducing hair loss have been described that include extracts from dong chong xia cho (U.S. Pat. No. 4,769,231); Berberis vulgaris or barberry (U.S. Pat. No. 4,769,231); saw palmetto berry (U.S. Pat. No. 5,750,108); Urtica dioica (U.S. Pat. No. 5,407,675); corncobs and aloe vera gel (U.S. Pat. No. 5,405,609); Berberis vulgaris or barberry (U.S. Pat. No. 5,607,693); Calanthe R. Br. or Phaius Lour. (U.S. Pat. No. 5,750,107); saw palmetto and African Pygeum (U.S. Pat. No. 5,972,345); Serenoa repents (U.S. Pat. No. 6,019,976); Vetiver grass (U.S. Pat. No. 6,193,976); hops, rosemary and Swertia (U.S. Pat. No. 6,447,762); Angelica and Astragali radix (U.S. Pat. No. 6,689,348); artemisia, parsley and crushed grapes (GB 2 060 378A), and Pterocarpus marsupium (U.S. Patent Application 20040146482).

A more complex composition comprising a combination of over 25 plant extracts is described in Japanese Patent Application JP60146829 for use as a hair tonic. U.S. Patent Application 20040156920 describes a general method for preparing oil extracts from Angiosperm and Gymnosperm plants and uses for these extracts, including application in disease resistance, stress resistance, general promotion of health and growth, delaying senescence, wound healing, skin repair, stimulation of hair growth, bone repair and lipid lowering.

U.S. Pat. No. 5,674,510 describes a hair treatment solution capable of reducing or eliminating alopecia and increasing hair growth that comprises garlic powder, brewer's yeast, grapefruit juice, acetic acid and kelp. U.S. Pat. No. 6,232,302 describes a method of increasing the percentage of hair in the hair growth cycle of the scalp by topically applying an effective amount of a depolymerized fucane sulphate obtained from seaweed, specifically from Fucus vesiculosus, Ascophyllum nodosum, Ecklonia kurome, Eisenia bicyclis, Laminaria digitata, Laminaria japonica, Padina pavonia, Pelvetia canaliculata, Sargassum linifolium or Undaria pinnalifda.

Fucus vesiculosus, commonly known as bladderwrack, is a type of kelp having a high iodine content, and containing various polysaccharides, such as fucoidans, which have been shown to have anti-thrombotic, anti-coagulant and wound-healing effects (Colliec, S., et al., Thromb. Res. 1991, 64:143-154; O'Leary, R., et al., Biol. Pharm. Bull., 2004, 27:266-270).

U.S. Pat. No. 6,103,272 describes a topical solution for promoting hair growth comprising colloidal silver and optionally aloe vera gel, allantoin, arnica flowers, comfrey leaves, horsetail herbal extract, jojoba, collogen, elastin, saponins, chamomile flowers, elkweed, jaborandi leaves, napca or rosemary leaves. Jaborandi is a general term used for various plants from the genus Pilocarpus, primarily with respect to the species: P. jaborandi, P. pennatifolius, P. trachylophus, P. microphyllus and P. spicatus. Extracts from jaborandi leaves have been used for many years as a herbal medicine for the treatment of various diseases and disorders (Lloyd, J. U. The Gleaner 1937, volume 46) and have shown some efficacy in treating skin disorders, such as eczema, pruritis, and psoriasis, as well as in darkening the colour of hair and in promoting the growth of the hair (see, King's American Dispensatory, Felter & Lloyd, 1898, available from Eclectic Medical Publications, Portland, Oreg.). One of the known active constituents of jaborandi is the alkaloid pilocarpine, which is used in the treatment of glaucoma and in ophthalmic settings for promoting constriction of the pupil of the eye. pilocarpine has also been used to treat xerostomia and related oral symptoms in patients with Sjogren's Syndrome (Vivino, F. B., 2001, Scandinavian Journal of Rheumatology, 30:1-39) In addition to pilocarpine, jaborandi contains a number of other alkaloids, as well as terpenes and tannic acids.

Holarrhena antidysynterica extracts are known in traditional herbal medicine for their ability to treat amoebic dysentery and other gastric disorders, such as diarrhea, indigestion, flatulence and colic (see, for example, Dictionary of Indian Medicinal Plants, Hussain, A., et al., 1992, Central Institute of Medicinal and Aromatic Plants, Lucknow, India). The bark of Holarrhena antidysynterica is also known to have an astringent effect. One of the active constituents of Holarrhena antidysynterica is thought to be conessine, a steroidal alkaloid that can be isolated from the bark of Holarrhena antidysynterica trees. More than thirty alkaloids have been isolated from Holarrhena antidysynterica, including conessine, kurchine, kuchicine, holarrhimine, conarrhimine, conaine, conessimine, iso-conessimine, conimine, holacetin, and conkurchin.

Rauwolfia serpentina (Indian snakewood) has been used for centuries in Ayurvedic and traditional Indian medicine as a febrifuge, a treatment for snake bites, diarrhea and dysentery, and for the relief of various central nervous system disorders. Rauwolfia alkaloids are used in the treatment of hypertension and severe agitation in patients with mental disorders.

Steroidal alkaloids are also produced by plants of the genus Veratrum (for example, jervine, rubijervine, pseudojervine and cyclopamine), Solanum (for example, solanine, tomatine, solasodine and solanidine), Buxus (for example, diacetylbuxadine, demethylcyclomikuranine, cyclomikuranine, cyclobuxophylline, buxaquamarine and spirofornabuxine) and Rauwolfia (for example, reserpine and rescinnamine). Many of these alkaloids are known toxins, including jervine and cyclopamine, which are teratogenic, and solanine, which is a mitotic poison. Jervine has also been shown to exert a teratogenic effect in several animal species. Defects were restricted to structures that depend upon normal chondrogenesis for their development. Jervine acts specifically during an early phase of die differentiation of mesenchyme into cartilage and it is likely that a specific stem cell population is the target tissue of this compound (Campbell et al., Dev. Biol. 1985; 111(2):464). Veratrum album is also known as an important weed on grazed montane grasslands, because it exhibits acute toxicity to mammals and locally displaces fodder plants (Schaffner et al., Biocontrol News and Information, 2002, 22:19 N-28N).

The use of steroidal alkaloids including solanidanes and C-nor-1)-homosteroidal alkaloids to reverse or inhibit multidrug resistance in cancer or bacterial, fungal, or parasite infections has been reported (United States Patent Application Publication No. 2003/0114393). The potent regulatory effects of jervine Veratrum alkaloids on hedgehog signalling, modulation of cholesterol biosynthesis and transport, and control of cell proliferation during mandibular arch morphogenesis have been reported. (Incardona et al., Development 1998; 125, 3553-3562, and Cooper et al. Science 5 Jun. 1998: Vol. 280. no. 5369, pp. 1603-1607).

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide a system and method for promoting hair growth and improving hair and scalp health.

In accordance with one aspect of the present invention, there is provided a system for promoting hair growth, said system comprising one or more of: an extract from a Veratrum plant, an extract from a Buxus plant, an extract from a Holarrhena plant, an extract from a Solanum plant, and an extract from a Rauwolfia plant.

In accordance with another aspect of the present invention, there is provided a system for promoting hair growth, said system comprising an extract from a Pilocarpus plant, a seaweed extract, and one or more of: an extract from a Veratrum plant, an extract from a Buxus plant, an extract from a Holarrhena plant, an extract from a Solanum plant, and an extract from a Rauwolfia plant.

In accordance with another aspect of the present invention, there is provided a method for promoting hair growth in a subject comprising the step of topically applying to the area where promotion of hair growth is desired an effective amount of an extract from a Pilocarpus plant, an effective amount of a seaweed extract and an effective amount of one or more of: an extract from a Veratrum plants an extract from a Buxus plant, an extract from a Holarrhena plant, an extract from a Solanum plant, and an extract from a Rauwolfia plant.

In accordance with another aspect of the present invention, there is provided a kit for promoting hair growth comprising the system according to the present invention and optionally instructions for use.

### BRIEF DESCRIPTION OF THE DRAWINGS

These and other features of the invention will become more apparent in the following detailed description in which reference is made to the appended drawings.

FIG. 1 depicts an example of the effect of the botanical compositions according to one embodiment of the present invention on hair growth in an affected area of a patient. FIG. 1A depicts the affected area before treatment and FIG. 1B depicts the affected area after treatment.

### DETAILED DESCRIPTION OF THE INVENTION

The present invention provides for a system for promoting hair growth comprising one or more plant extracts. In its simplest embodiment, the system comprises one or more plant extracts that are derived from a steroidal alkaloid-containing plant selected from: a Veratrum plant, a Buxus plant, a Holarrhena plant, a Solanum plant, and a Rauwolfia plant. In other embodiments, the system also includes an extract from a Pilocarpus plant and/or a seaweed extract. The extracts can be crude plant extracts, substantially pure extracts, extracts enriched in phytochemicals such as alkaloids, glycosides and/or polysaccharides, or substantially purified phytochemicals such as alkaloids, glycosides and/or polysaccharides extracted from said plants. In general, the plant extracts are included in the system in the form of compositions comprising the plant extract(s), which are referred to herein as “botanical compositions.” The plant extracts of the system can be combined and provided as a single botanical composition, or each extract can be provided and maintained as a separate botanical composition. Alternatively, two or more of the plant extracts of the system can be combined and provided as a first botanical composition, along with one or more additional plant extracts of the system which are provided and maintained as separate compositions. When the system comprises more than one of a Veratrum plant extract, a Buxus plant extract, a Holarrhena plant extract, a Solanum plant extract, and a Rauwolfia plant extract, these extracts can be provided as a single botanical composition or as separate botanical compositions.

The system is effective in promoting the growth of hair and can, therefore, be used by subjects to promote hair growth in regions of the body where hair growth has ceased or diminished, or where hair growth is naturally sparse. The system can also be used to reduce hair loss, enhance or restore hair colour, increase the thickness of hair, improve the general appearance of hair, and/or reduce or eliminate dandruff.

The present invention also provides for methods of using the system to promote hair growth in a subject. Methods of reducing hair loss, enhancing or restoring hair colour, increasing the thickness of hair, improving the general appearance of hair, and/or reducing or eliminating dandruff in a subject using the system of the present invention are also provided.

The system and methods of the present invention can be employed, for example, by subjects experiencing complete or partial hair loss, such as that encountered in various forms of alopecia, including alopecia androgenetica (“male pattern baldness”), alopecia cicatrista and alopecia areata, or as a result of trauma, injury, chemotherapy, stress, genetic factors, hormonal changes, disease, nutritional imbalance, scalp abnormalities and the like, as well as for subjects with naturally sparse hair growth.

The system and methods of the present invention are suitable for use in both humans and other mammalian species. For example, the system and methods can be applied to non-human mammals used in wool or fur production to accelerate hair growth thereby permitting, for example, greater net annual wool production or reducing the time needed to produce mature pelts.

### DEFINITIONS

Unless defined otherwise, all technical and scientific terms used herein have the same meaning as commonly understood by one of ordinary skill in the art to which this invention belongs.

The term “plant,” as used herein, is intended to include both terrestrial and aquatic plants.

The term “seaweed,” as used herein, refers to a marine algae from the family Phaeophyceae, Rhodophyceae or Chlorophyta.

The term “plant material,” as used herein, refers to a part or parts of a plant taken either individually or in a group. Examples include, but are not limited to, bulbs, leaves, flowers, fruits, rhizomes, roots, seeds, seed pods, stems, fronds, bark, branchlets, twigs and other parts of a plant.

The term “extract” or “plant extract,” as used herein, refers to a preparation derived from plant material that is in a different form than the original plant material from which it is derived. An extract can be as simple as mechanically lysed cells, in which case the preparation may be clarified by centrifugation or filtration to remove insoluble debris, or it can be a preparation derived by contacting plant material with one or more solvents. The term “extract” also encompasses preparations that undergo one or more purification steps to enrich the content of phytochemicals, such as alkaloids, glycosides and/or polysaccharides, as well as preparations comprising partially or substantially purified phytochemicals derived from the plant material.

The terms “steroid” and “steroid-like” are used interchangeable herein and refer to a general class of polycyclic compounds possessing the skeleton of cyclopentanophenanthrene or a skeleton derived there from by one or more bond scissions or ring expansions or contractions. The rings may be substituted at one or more positions, to create derivatives that adhere to the rules of valence and stability, such as by methyl or other lower alkyl groups, hydroxyl groups, alkoxyl groups and the like.

The term “stressor,” as used herein, refers to a factor, such as a physical stress, a chemical compound, or a biological agent that is applied to a plant in order to increase the production of certain phytochemicals prior to harvesting the plant.

The term “subject,” as used herein, refers to a mammal in need of treatment or who would otherwise benefit from the use of the system of the invention.

As used herein, the term “about” refers to a +/−10% variation from the nominal value. It is to be understood that such a variation is always included in any given value provided herein, whether or not it is specifically referred to.

### System Comprising Plant Extracts

The system of the present invention comprises one or more extracts from steroidal alkaloid-containing plants selected from a Veratrum plant, a Buxus plant, a Holarrhena plant, a Solanum plant and a Rauwolfia plant. In one embodiment, the system comprises one or more extracts from a Veratrum plant and/or a Buxus plant. In addition, the system can comprise one or more extracts from a Pilocarpus plant, and/or one or more seaweed extracts. The system can further comprise additional plant extracts that provide additional beneficial effects to the subject being treated.

The plant extracts in the system can be provided in the form of botanical compositions, which optionally comprise a suitable carrier, diluent and/or excipient and which can further optionally comprise other components that supplement, facilitate or otherwise improve their efficacy. Such additional components can be antimicrobial agents, moisturising agents, vitamins, minerals, and the like.

Without being limited to any theory or mechanism, it is believed that the plant extracts included in the system of the present invention help to increase the stem cell population of the hair, increase production of the structural protein keratin, open skin pores and/or provide essential nutrients to the hair, thereby promoting growth of the hair and improving its overall health. In one embodiment of the invention, the system comprises an extract from a Pilocarpus plant for facilitating the opening of skin pores; one or more extracts from steroidal alkaloid-containing plants for facilitating an increase in the stem cell population of hair and/or a down-regulation of the conversion of testosterone to dihydrotestosterone; and a seaweed extract for providing nutrients to the hair.

In one embodiment of the present invention, the system comprises one or more extracts from steroidal alkaloid-containing plants selected from Veratrum plants, Buxus plants and Holarrhena plants; in combination with an extract from a Pilocarpus plant, and a seaweed extract. In another embodiment, the system comprises one or more extracts from steroidal alkaloid-containing plants selected from Veratrum plants and Buxus plants; in combination with an extract from a Pilocarpus plant, and a seaweed extract. In a further embodiment, the system comprises one or more extracts from steroidal alkaloid-containing plants selected from Buxus plants and Holarrhena plants; in combination with an extract from a Pilocarpus plant, and a seaweed extract.

In an alternative embodiment of the present invention, the system comprises one or more extracts from Veratrum plants, an extract from a Pilocarpus plant and a seaweed extract. In another embodiment, the system comprises one or more extracts from Buxus plants, an extract from a Pilocarpus plant and a seaweed extract. In another embodiment, the system comprises one or more extracts from Holarrhena plants, an extract from a Pilocarpus plant and a seaweed extract. In a further embodiment, the system comprises one or more extracts from Solanum plants, an extract from a Pilocarpus plant and a seaweed extract. In another embodiment, the system comprises one or more extracts from Rauwolfia plants, an extract from a Pilocarpus plant and a seaweed extract.

In another embodiment, the system comprises one or more extracts from steroidal alkaloid-containing plants selected from Veratrum plants, Buxus plants, Holarrhena plants and Rauwolfia plants; in combination with an extract from a Pilocarpus plant, and a seaweed extract. In another embodiment the system comprises one or more extracts from steroidal alkaloid-containing plants selected from Veratrum plants, Rauwolfia plants, and Buxus plants; in combination with an extract from a Pilocarpus plant and a seaweed extract. In a further embodiment, the system comprises one or more extracts from steroidal alkaloid-containing plants selected from Buxus plants, Rauwolfia plants and Holarrhena plants; in combination with an extract from a Pilocarpus plant and a seaweed extract. In another embodiment, the system comprises one or more extracts from steroidal alkaloid-containing plants selected from Veratrum plants and Rauwolfia plants; in combination with an extract from a Pilocarpus plant and a seaweed extract.

### 1. Plant

### Steroidal Alkaloid-Containing Plants

The system of the present invention comprises one or more extracts from steroidal alkaloid-containing plants selected from Veratrum plants, Buxus plants, Holarrhena plants, Solanum plants, and Rauwolfia plants. The proportion of total plant material used to prepare the extracts for the system of the present invention that is derived from these steroidal alkaloid-containing plants is between about 15% and about 95% w/w. In one embodiment, the proportion of steroidal alkaloid-containing plant material is between about 20% and about 90% w/w of the total plant material used to prepare the extracts for the system. In another embodiment, the proportion of steroidal alkaloid-containing plant material is between about 25% and about 90% w/w of the total plant material used to prepare the extracts for the system. In a further embodiment, the proportion of steroidal alkaloid-containing plant material is between about 30% and about 85% w/w of the total plant material used to prepare the extracts for the system.

In an alternate embodiment of the present invention, the proportion of steroidal alkaloid-containing plant material is between about 30% and about 60% w/w of the total plant material used to prepare the extracts for the system. In a further embodiment, the proportion of steroidal alkaloid-containing plant material is between about 30% and about 50% w/w of the total plant material used to prepare the extracts for the system.

### Veratrum Plants

The Veratrum plant extract(s) included in the system of the present invention can be derived from a variety of known Veratrum species and subspecies. Suitable examples include, but are not limited to, Veratrum album white hellebore), Veratrum californicum (California false hellebore), Veratrum grandiflorum, Veratrum japonicum, Veratrum nigrum (Black false hellebore), Veratrum officinale (Sabadilla), Veratrum sabadilla (Sabadilla), Veratrum spp. (False hellebore), Veratrum viride (Green hellebore) and Veratrum woodii.

In one embodiment, the system of the present invention includes a Veratrum plant extract derived from Veratrum album, Veratrum californicum, Veratrum japonicum, Veratrum nigrum or Veratrum viride.

### Buxus Plants

The Buxus plant extract(s) included in the system of the present invention can be derived from a variety of known Buxus species and subspecies. Suitable examples include, but are not limited to, Buxus acuminata, Buxus balearica, Buxus bodineri, Buxus citrifolia, Buxus crassifolia, Buxus cochinchensis, Buxus cubana, Buxus foliosa, Buxus harlandii, Buxus hildebrandtii, Buxus hyrcana, Buxus macrophylla, Buxus macowani, Buxus madagascarica, Buxus megistophylla, Buxus mexicana, Buxus microphylla (including Buxus microphylla japonica, Buxus microphylla koreana and Buxus microphylla sinica), Buxus papillosa, Buxus portoricensis, Buxus pubescens, Buxus revoluta, Buxus riparia, Buxus rotundifolia, Buxus rugulosa, Bixis rupicola, Buxus sinica, Buxus sempervirens (Box), Buxus suffructaca (Dwarf Box), Buxus vaccinioides, Buxus rivularis, Buxus rolfei and Buxus wallichiana.

In one embodiment, the system of the present invention includes a Buxus plant extract derived from Buxus balearica, Buxus bodineri, Buxus harlandii, Buxus microphylla (including Buxus microphylla japonica, Buxus microphylla koreana and Buxus microphylla sinica), Buxus riparia, Buxus rugulosa, Buxus sinica, Buxus sempervirens (Box) and Buxus wallichiana. In another embodiment of the present invention, system of the present invention includes a Buxus plant extract derived from Buxus sempervirens. In another embodiment, the Buxus plant extract is derived from the leaves of the Buxus plant.

### Holarrhena Plants

The Holarrhena plant extract(s) included in the system of the present invention can be derived from a variety of known Holarrhena species and subspecies. Suitable examples include, but are not limited to, Holarrhena antidysenterica, Holarrhena febrifuga, Holarrhena floribunda and Holarrhena pubescens.

In one embodiment, the system of the present invention includes a Holarrhena plant extract derived from the bark of the Holarrhena plant.

### Solanum Plants

The Solanum plant extract(s) included in the system of the present invention can be derived from a variety of known Solanum species and subspecies. Suitable examples include, but are not limited to, Solanum americanum, Solanum aculeatissimum (Loveapple), Solanum capsicastrum, Solanum carolinense (Horsenettle), Solanum citrullifolium, Solanum dulcamara (Woody Nightshade), Solanum elaeagnifolium, Solanum erianthum (Tobacco Nightshade), Solanum heterodoxum, Solanum integrifolium (Ruffled Red Eggplant), Solanum laciniatum (Tasmanian Kangaroo Apple), Solanum luteum, Solanum melanocerasum (Sunberry), Solanum mammosum (Apple of Sodom), Solanum melongena (Eggplant), Solanum nigrum (Black Nightshade), Solanum oleraceum (Jagueribo), Solanum physalifolium, Solanum pseudo-capsicum (Jerusalem Cherry), Solanum quitoense (Naranjilla), Solanum rostratum, Solanum sarrachoides, Solanum sisymbrifolium, Solanum sodomaoum (Apple of Sodom), Solamum triflorum, Solanum tuberosum (Potato) and Solanum xanthocarpum (Kantikari).

In one embodiment, the system of the present invention includes a Solanum plant extract derived from Solanum dulcamara (Woody Nightshade). In another embodiment, the system of the present invention includes a Solanum plant extract derived from the aboveground parts of the plant.

### Rauwolfia Plants

The Rauwolfia plant extract(s) included in the system of the present invention can be derived from a variety of known Rauwolfia species and subspecies. Suitable examples include, but are not limited to, Rauwolfia serpentina, Rauwolfia vomitoria, Rauwolfia canescens and Rauwolfia tetraphylla.

In one embodiment, the system of the present invention includes a Rauwolfia plant extract derived from Rauwolfia serpentina.

### Pilocarpus Plants

The Pilocarpus plant extract(s) included in the system of the present invention can be derived from a variety of known Pilocarpus species or subspecies. Suitable examples include, but are not limited to, Pilocarpus cearensis, Pilocarpus jaborandi (also known as: Pernambuco jaborandi), Pilocarpus microphyllus (also known as: Maranham jaborandi), Pilocarpus officinalis, Pilocarpus pauciflorus (a subspecies of Pilocarpus spicatus), Pilocarpus pennatifolius (including Pilocarpus pennatifolius jaborandi), Pilocarpus racemosus (also known as: Guadeloupe jaborandi), Pilocarpus spicatus (also known as: Aracati jaborandi) and Pilocarpus trachylophus.

In one embodiment, the system of the present invention includes a Pilocarpus plant extract derived from Pilocarpus pennatifolius, Pilocarpus jaborandi or Pilocarpus microphyllus. In another embodiment, the system of die present invention includes a Pilocarpus plant extract derived from the leaves and fine sterns of the Pilocarpus plant.

In one embodiment, the system of the present invention includes an extract from a Pilocarpus plant indigenous to Brazil. In another embodiment the system of the present invention includes an extract from a Pilocarpus plant indigenous to the Brazilian provinces of Ceara or Maranhao.

The proportion of total plant material used to prepare the extracts for the system of the present invention that is derived from the Pilocarpus plant(s) is between about 3% and about 20% w/w. In one embodiment, the proportion of Pilocarpus plant material is between about 5% and about 18% w/w of the total plant material used to prepare the extracts for the system. In another embodiment, the proportion of Pilocarpus plant material is between about 7% and about 15% w/w of the total plant material used to prepare the extracts for the system.

### Seaweed

The seaweed from which the seaweed extract component of the system of the present invention is derived can be a brown seaweed (Phaeophyceae), a red seaweed (Rhodophyceae) or a green seaweed (Chlorophyta). Exemplary brown seaweeds (or kelps) include, but are not limited to Fucus, Laminara (for example, Laminaria digitata, Laminaria saccharina or Laminaria japonica), Sargassum (Sargassum natan or Sargassum fluitan), Ascophyllum (for example, Ascophyllum nodosum) and Ecklonia species. Exemplary red seaweeds include, but are not limited to Porphyra and Chondrus species, for example, Chondrus crispus. Exemplary green seaweeds include, but are not limited to, Ulva species, for example, Ulva latuca.

In one embodiment of the invention, she system comprises a seaweed extract from a seaweed which produces organic iodine. In another embodiment of the present invention, the seaweed extract is derived from a brown seaweed. In another embodiment, the seaweed extract is derived from one of a variety of known Fucus species and subspecies. Suitable examples include, but are not limited to, Fucus amylaceus (Ceylon Moss), Fucus canaliculatus (Wrack), Fucus digitatus, Fucus gardneri, Fucus helminthocorton (Corsican Moss), Fucus natans (Gulf-Weed), Fucus nodosus (Knobbed Wrack), Fucus serratus (Black Wrack), Fucus siliquosus (Wrack), Fucus spiralis and Fucus vesiculosus (bladder-wrack).

In a specific embodiment of the present invention, the system comprises a seaweed extract derived from Fucus digitatus, Fucus helminthocorton, Fucus natans, Fucus nodosus, Fucus serratus, Focus siliquosus, Fucus spiralis and Fucus vesiculosus. In a further embodiment, the seaweed extract is derived from Fucus vesiculosus.

The proportion of total plant material used to prepare the extracts for the system of the present invention that is derived from seaweed is between about 5% and about 55% w/w. In one embodiment, the proportion of seaweed plant material is between about 7% and about 55% w/w of the total plant material used to prepare the extracts for the system. In another embodiment, the proportion of seaweed plant material is between about 10% and about 50% w/w of the total plant material used to prepare the extracts for the system.

### 2. Preparation of Extracts

Plant material is obtained from the selected plants by standard techniques. The plant material employed in the preparation of the extracts can be the entire plant, or it can be one or more distinct parts of the a plant, for example, leaves, seeds, roots, stems, flowers, fronds, bark, branches, twigs, or various combinations thereof.

The plant extracts of the present invention can be prepared from plant material harvested from unstressed (“natural”) plants or from plants that have been treated with one or more stressor prior to harvest. The stressor can be a chemical stressor or a physical stressor. Examples of chemical stressors include, but are not limited to, organic and inorganic acids, fatty acids, glycerides, pitospholipids, glycolipids, organic solvents, amino acids and peptides, monosaccharides, oligosaccharides, polysaccharides and lipopolysaccharides, phenolics, alkaloids, terpenes and terpenoids, antibiotics, detergents, polyamines, peroxides, and ionophores. Examples of physical stressors include, but are not limited to, ultraviolet radiation, low temperature, high temperature, osmotic changes (for example, induced by salt or sugars), and nutritional deprivation (for example, depriving the plant of an essential nutrient, such as nitrogen, phosphorus or potassium).

The extracts can be prepared using fresh plant material or the plant material can be treated, for example, by drying, freezing, lyophilising, or some combination thereof, prior to preparation of the extracts, The plant material can be used immediately after harvest or it can be stored for a period of time before preparation of the extract. If desired the plant material can undergo one or more of the above treatments prior to storage.

Plant material from one or more of the selected plants can be combined prior to preparation of the extract, or separate extracts can be prepared from each individual plant and either combined at a later stage or maintained as separate extracts. In one embodiment of the present invention, the plant material from at least two of the selected plants is combined prior to preparing the extract. In another embodiment, the plant material from all the selected plants is combined prior to preparing the extract. In still another embodiment of the invention, the plant material from the selected plants is not combined prior to preparing the extracts, and the extracts are maintained as separate extracts.

As indicated above, the plant material used to prepare the extracts can be fresh, dried or frozen. The extract can be prepared by simply crushing or fragmenting the plant material. For example, the plant material can be pounded, crushed or sliced mechanically, using a grinder, hammer mill, knife mill, tooth mill, blender, pestle and mortar, or other device to fragment the plant parts into small pieces or particles, or the plant material can be frozen in liquid nitrogen and then crushed or fragmented into smaller pieces. Other size reduction methods known in the art can also be used.

Alternatively, the plant extracts can be prepared by contacting the plant material with one or more solvents. If desired, the plant material can be crushed or fragmented as described above prior to being contacted with said solvent(s) in order to present a greater surface area to the solvent. The plant material can be crushed or fragmented under pressure, if desired, in order to provide a greater surface area for subsequent solvent contact.

In one embodiment of the present invention, the plant extract is prepared by contacting the plant material with one or more solvents. In another embodiment, the plant material is crushed or fragmented prior to being contacted with the one or more solvents.

### Solvents

When a solvent is used to prepare the extract, the solvent can be an aqueous solvent, an organic solvent, an aqueous-organic mixture, or a mixture of two or more organic solvents. Aqueous solvents suitable for use in the preparation of the extracts include, but are not limited to, water, various aqueous buffers and solutions of organic and/or inorganic salts. The pH of the aqueous solutions can be adjusted to a suitable value by addition of acids and bases as is known in the art and can range from a pH of between about 2 and about 12. Suitable organic solvents include, various natural oils, primary alcohols, such as methyl alcohol (methanol), ethyl alcohol (ethanol), 1-propanol and 1-butanol; secondary alcohols such as 2-propanol and 2-butanol; tertiary alcohols such as 2-methyl-2-propanol; liquid polyhydric alcohols such as glycerine and glycols; and other known organic solvents such as acetone, tetrahydrofuran, acetonitrile, 1,4-dioxane, pyridine, dimethylsulfoxide, N,N-dimethyl formamide, diethyl ether, hexane, heptane, dichloromethane and ethyl acetate, or a combination of the above solvents.

Exemplary oils that can be used as solvent include, but are not limited to, vegetable oils, such as almond, anise, balm, bay, bergamot, borage, cajeput, canola, castor, cedarwood, cinnamon, clove, coconut, corn, cottonseed, evening primrose, flaxseed, grape seed, hempnut, jojoba bean, Karanj (Pongamia glabra), lavender, linseed, macadamia, mustard, Neem (Azadirachta indica), olive, orignaum (thyme), peanut, rapeseed, safflower, sesame, soybean, sunflower, Tea Tree, walnut and wheat germ oil, or mineral oils, such as liquid paraffin, or a combination of any of the above.

Suitable glycols include, for example, ethylene glycol, propylene glycol, diethylene glycol, dipropylene glycol and 1,3-butylene glycol.

In one embodiment of the present invention, the solvent comprises an aqueous solvent, a lower alcohol, a natural oil, or a combination thereof. In the context of the present invention, a “lower alcohol” refers to an alcohol having 1 to 4 carbon atoms, such as a primary, secondary, tertiary or liquid polyhydric alcohol. In another embodiment, the lower alcohol is selected from the group of: methyl alcohol (methanol), ethyl alcohol (ethanol), 1-propanol, 1-butanol, 2-propanol, 2-butanol, 2-methyl-1-propanol, 2-methyl-2-propanol, glycerine, ethylene glycol, propylene glycol, diethylene glycol, dipropylene glycol and 1,3-butylene glycol. In a further embodiment, the solvent comprises a vegetable oil.

Aqueous-organic mixtures generally comprise a ratio of organic solvent(s):aqueous solvent(s) of about 2:1 to 1:20. In one embodiment, the solvent used in the preparation of the extracts is an aqueous-organic mixture comprising a ratio of organic solvent(s):aqueous solvent(s) of about 1:1 to 1:15. In another embodiment, the solvent used in the preparation of the extracts is an aqueous-organic mixture comprising a ratio of organic solvent(s):aqueous solvent(s) of about 1:2 to 1:10.

### Extraction

As a first step in the extraction process, the plant material and solvent(s) are combined and mixed thoroughly.

The plant material and solvent(s) are combined in a ratio of between about 10:1 to about 1:100 w/w plant material:solvent(s) and, more typically in a ratio of between about 5:1 to about 1:70 w/w plant material:solvent(s). In one embodiment, the ratio is between about 3:1 to about 1:60 w/w plant material:solvent(s). In another embodiment, the ratio is between about 1:2 to about 1:50 w/w plant material:solvent(s). In another embodiment, ratio is between about 1:1 to about 1:50 w/w plant material:solvent(s). In a further embodiment, the ratio is between about 1:1 to about 1:30 w/w plant material:solvent(s). In a still further embodiment, the ratio is between about 1:1 to about 1:10 w/w plant material:solvent(s).

In an alternative embodiment of the present invention, the amount of plant material employed in the initial extraction step is between about 1% to about 50% w/w.

The overall extraction process can comprise a single extraction step, or it can comprise multiple (i.e. two or more) extraction steps. Typically, each extraction step comprises contacting the plant material with a solvent with adequate mixing over a period of time selected as known in the art, depending on known factors, such as the starting material, the extraction process, the extraction temperature, the ratio of solvent to plait material, and the like.

Various extraction methods known in the art can be employed and may entail, for example, one or more of maceration, remaceration, digestion, agitation, agitation maceration, filtration, vortex extraction, centrifugation, ultrasonic extraction, countercurrent extraction, percolation, repermolation, evacolation (extraction under reduced pressure), diacolation and solid liquid extraction under continuous reflux in a Soxhlet extractor (see, for example, in Hagers Handbuch der Pharmazeutischen Praxis, 5^{th} Edition, Vol. 2 pp. 1026-1030, Springer Verlag, Berlin-Heidelberg-New York 1991). Percolation may be suitable when preparing extracts on a large-scale.

The plant material is left in contact with the solvent(s) over a period of time sufficient to ensure adequate exposure of the plant material to the solvent(s) such that active components from the plant material are taken up by the solvent(s). Typically this period of time is between about 1 hour and 4 months, although one skilled in the art will appreciate that longer or shorter times may be appropriate. The solvent can be heated prior to contacting the plant material, for example, to a temperature between about 10° C. and about 150° C. prior to being added to the plant material. Alternatively, the plant material can be boiled gently in the solvent for a short period of time and then allowed to cool. The extraction step is generally conducted at a temperature between about 4° C. and about 65° C. In one embodiment, the extraction is carried out at a temperature between about 10° C. and about 60° C. In another embodiment, the extraction is carried out between about 20° C. and about 60° C. In a further embodiment, the extraction is carried out between about 25° C. and about 50° C. The extraction process can be carried out in a normal atmosphere, or it can be carried out in an inert gas atmosphere when oxidation of the ingredients of the extract is a concern. This may be useful, for example, where the extraction is carried out at temperature above 40° C.

In one embodiment of the present invention, the extraction procedure is conducted over a period of time between about 1 hour and about 4 months at a temperature between about 4° C. and about 50° C. As indicated above, the solvent can be heated prior to being combined with the plant material for the extraction process Adequate contact of the plant material with the solvent can be encouraged by shaking or otherwise agitating the suspension either periodically or continuously. In one embodiment of the present invention, the extraction is carried out in the dark.

The liquid extract is then separated from the solid (insoluble) matter. This separation can be achieved by one or more standard processes known to those skilled in the art, such as filtration (regular, suction, vacuum or under pressure), ultrafiltration, centrifugation, ultracentrifugation, or other means known in the art to separate solids from a solution. If required, the solid material (or marc) thus obtained can be pressed and the resulting liquid added to the extract.

When further extraction step(s) are to be carried out, the solid material (or mare) can be recovered and submitted to one or more additional extractions as described above, and the extracts combined. The marc can be used “as is” or it can be incinerated or calcined prior to the additional extraction(s). Typically, for calcinations, the pressed marc is spread out in a fireproof dish and steadily heated from below. In general, the temperature for the calcination process is kept below about 400° C., to avoid melting and fusing of the resultant salt. The calcination will usually proceed in stages and pass through different colour changes, the first change being the carbonisation of the mare, which is black, then through different shades of brown and orange, then finally grey and then white. The salts/ashes obtained from the calcination process can be added back to the initial extract and a subsequent extraction conducted using the initial liquid extract as solvent, or the salts can be added to a new solvent and a further extraction carried out, and the extract obtained added to the initial extract.

In one embodiment, the salts/ashes obtained from the calcination process comprise an oligo form of one or more phytochemicals.

It is also contemplated that additional ingredients may be added to the mixture of the plant material and solvents, for example, to improve the quality of the extract. These additional ingredients may include acids, such as nitric acid. These additional ingredients may be added to either the solvent at the time of first maceration of the plant material, or they may be added to the mixture of the plant material and solvent. The amount of these additional ingredients may vary between 0.1% and 5% of the volume of the solvent added to the plant material. In one embodiment of the invention, 1% v/v nitric acid is added to the solvent. In another embodiment, nitric acid is added to the solvent at the time of first maceration.

In one embodiment of the present invention, the extracts are prepared by crushing or fragmenting the plant material to provide a pulp, then adding solvent to the pulp in a ratio of about 2:1 to about 1:20 w/w plant material:solvent and mixing thoroughly. The mixture is allowed to stand for between about 1 hour to 100 days in a dark and cool environment. The mixture is shaken frequently to ensure complete extraction of the relevant components from the plant material into the solvent. After the extraction period, the suspension can be left for an additional time of between about 30 minutes to 10 days to allow the larger solid particles to settle at the bottom of the extraction vessel. The solid material is then removed to yield the liquid plant extract.

In another embodiment of the present invention, the extracts are prepared by crushing or fragmenting the plant material to provide a pulp, then mixing the pulp with an oil in a ratio between about 2:1 to about 1:50 w/w plant material:oil. The temperature of the oil used in this step is between about 10° C. to about 150° C. Alternatively, the plant material may be simmered gently for between about one minute and 2 hours after being dispersed in the oil. In a specific embodiment, the plant material may be simmered gently for between about one and sixty minutes after being dispersed in the oil. In one embodiment, the oil/pulp mixture is then mixed with liquid paraffin in a ratio of between 3:1 and 1:3 w/w plant material:liquid paraffin, for example, 2:3 w/w plant material:liquid paraffin, followed by mixing with about 0.2% to 2% w/w of the same or a different oil. The final mixture is allowed to stand for between about 7 to 10 days in dark and cool place with shaking twice daily to ensure complete extraction of the relevant components from the plant material into the oil. After the extraction period, the solid material can be removed if desired, for example, by filtration.

In a further embodiment of the present invention, the extracts are prepared by crushing or fragmenting the plant material to provide a pulp, then mixing with an aqueous solvent in a ratio between 1:2 to 1:20 w/w. The temperature of the aqueous solvent used in this step is between about 10° C. to about 150° C. Alternatively, the plant material may be simmered gently for between about one minute and 2 hours after being dispersed in the aqueous solvent. In a specific embodiment, the plant material may be simmered gently for between about one and sixty minutes after being dispersed in the aqueous solvent. In one embodiment, the aqueous solvent/pulp mixture is then mixed with an organic solvent in a ratio between about 1:1 to about 1:20 w/w plant material:solvent. A second organic solvent can be added at this stage, if desired, in an amount between about 0.2% to about 20% w/w. The mixture is allowed to stand for a time between about 1 hour and 100 days in a dark and cool environment with frequent shaking to ensure complete extraction of the relevant components from the plant material into the solvent. After the extraction period, the suspension can be left for an additional time of between about 30 minutes to 10 days to allow larger solid particles to settle at the bottom of the vessel and, if desired, the solid material can be removed, for example, by filtration.

In another embodiment of the invention, the extracts are prepared by crushing or fragmenting the plant material to provide a pulp, then mixing with a solvent in a ratio between about 1:2 to about 1:10 w/w. The mixture is then incubated for between about 21 days to 100 days in the dark at about 30° C. to about 40° C. with frequent shaking to ensure complete extraction of the relevant components from the plant material into the solvent. After the extraction period, the suspension can be left for an additional time of between about 30 minutes to 10 days to allow larger solid particles to settle at the bottom of the vessel. If desired, the solid material can then be removed, for example, by filtration.

In one embodiment of the present invention, the solvent is an organic solvent. In another embodiment, the solvent is an aqueous-organic mixture. In a further embodiment, the solvent is an aqueous-organic mixture having a ratio of organic solvent:aqueous solvent of about 1:2 to 1:10.

In a specific embodiment of the present invention, after the initial extraction, the solid material is removed (for example, by filtration) and subjected to a calcinations process. In another embodiment, the calcined solid material is added back to the initial liquid extract and subjected to a further extraction. In a further embodiment, the calcined material and initial liquid extract are combined and incubated for between about 21 days and 100 days in the dark at a temperature of about 30° C. to about 40° C., with frequent shaking. In another embodiment, about 2% to about 20% propylene glycol is added to the combined calcined material and initial extract prior to the second extraction. The plant extract can then be further incubated without shaking for about 30 minutes to about 30 days to allow any solid material to settle. In one embodiment, the plant extract is incubated for 21 days to allow solid material to settle. The solid material can be removed, for example, by filtration, if desired.

The present invention also contemplates that other methods known in the art may be employed to prepare the plant extracts, for example, the method as generally described in U.S. Patent Application 20040156920.

After the extraction process, extracts may be concentrated, if desired, prior to being used in the botanical compositions by removing some or a substantial portion of the solvent and/or water. The extracts may also be fractionated, using methods common to those of skill in the art (such as a second extraction, filtration, size fractionation by gel filtration or gradient centrifugation, etc.), in order to provide extracts enriched in phytochemicals, such as alkaloids, glycosides and/or polysaccharides and/or submitted to a decolourisation process. If desired, the extracts thus prepared may be subjected, for example, to the selective removal of individual unwanted ingredients. In one embodiment, the extracts have a final solids content of about 5% to about 10% by weight and are used “as is.” In another embodiment, the solvent is substantially or completely removed by drying, for example, by spray or freeze drying.

### Substantially Purified Phytochemicals

The present invention also provides for extracts that comprise substantially purified alkaloids, glycosides and/or polysaccharides derived from the plant material. Such extracts are initially prepared as described above and then subjected to one or more additional purification steps. There are a number of techniques well known in the art for enriching active components in complex mixtures that may be employed in the context of the present invention. Examples of these techniques include, but are not limited to, solid-liquid extraction, liquid-liquid extraction, solid-phase extraction (SPE), membrane filtration, ultrafiltration, dialysis, electrophoresis, solvent concentration, centrifugation, ultracentrifugation, liquid or gas phase chromatography (including size exclusion, affinity, and the like), lyophilisation, evaporation, precipitation with various “carriers” (including PVPP, carbon, antibodies, and the like), or various combinations thereof. One skilled in the art would appreciate bow to use such options, in a sequential fashion, in order to enrich each successive fraction in the phytochemicals of interest.

Solid-liquid extraction includes the use of soxhlet extractors, vortex shakers, ultrasounds and other means to enhance extraction, as well as recovery by filtration, centrifugation and related methods as described in the literature (see, for example, R. J. P. Cannell, Natural Products Isolation, Humana Press, 1998). Examples of solvents that may be used include, but are not limited to, hydrocarbon solvents, chlorinated solvents, organic esters, organic ethers, alcohols, water, and mixtures thereof.

Liquid-liquid extraction includes the use of various mixtures of solvents known in the art, including solvents under supercritical conditions. Typical solvents include those listed above. The liquid-liquid extraction can be effected manually, or it can be semi-automated or completely automated, and the solvent can be removed or concentrated by standard techniques in the art (see, for example, S. Ahuja, Handbook of Bioseparations, Academic Press, 2000).

Solid-phase extraction (SPE) techniques include the use of cartridges, columns or other devices known in the art. The sorbents that may be used with such techniques include, but are not limited to, silica gel (normal phase), reverse-phase silica gel (modified silica get), ion-exchange resins, and fluorisil. The invention also includes the use of scavenger resins or other trapping reagents attached to solid supports derived from organic or inorganic macromolecular materials to enrich the desired phytochemical content of the extracts.

Membrane, reverse osmosis and ultrafiltration includes the use of various types of membranes known in the art, as well as the use of pressure, vacuum, centrifugal force, and/or other means that can be utilised in membrane and ultrafiltration processes (see, for example, S. Ahuja, Handbook of Bioseparations. Academic Press, 2000).

Dialysis can be conducted using membranes having a molecular weight cut-off varying from less than about 0.5 KDa to greater than about 50 KDa. Extracts enriched in the selected phytochemicals can be recovered from either the dialysate or the retentate by various means known in the art including, but not limited to, evaporation, reduced pressure evaporation, distillation, vacuum distillation, and lyophilization.

Chromatography can be conducted by various techniques known in the art and described in the literature (see, for example, G. Sofer, L. Hagel, Handbook of Process Chromatography, Academic Press, 1997). Examples include, but are not limited to, regular column chromatography, flash chromatography, high performance liquid chromatography (HPLC), medium pressure liquid chromatography (MPLC), supercritical fluid chromatography (SFC), countercurrent chromatography (CCC), moving bed chromatography, simulated moving bed chromatography, expanded bed chromatography, and planar chromatography. Examples of sorbents that may be used in the above chromatographic methods include, but are not limited to, silica gel, alumina, fluorisil, cellulose and modified cellulose, various modified silica gels, ion-exchange resins, size exclusion gels and other sorbents known in the art (see, for example, T. Hanai, HPLC: A Practical Guide, RSC Press, UK 1999). The present 11 invention also includes the use of solvent gradients to effect the fractionation, partial purification, and/or purification of the active ingredients by chromatographic methods. Examples of solvents that may be utilised include, but are not limited to, hexanes, heptane, pentane, petroleum ethers, cyclohexane, heptane, diethyl ether, methanol, ethanol, isopropanol, propanol, butanol, isobutanol, tert-butanol, water, dichloromethane, dichloroethane, ethyl acetate, tetrahydrofuran, dioxane, tert-butyl methyl ether, acetone, and 2-butanone. When water or an aqueous phase is used, it may contain varying amounts of inorganic or organic salts, and/or the pH may be adjusted to different values with an acid or a base such that fractionation and/or purification is enhanced.

Selective precipitation includes the use of various solvents and solvent combinations, the use of temperature changes, the addition of precipitant and/or modifiers, and/or modification of the pH by addition of base or acid to effect a selective precipitation of phytochemicals.

The invention also includes the isolation of phytochemicals by steam distillation, hydrodistillation, or other related methods of distillation known in the art (see, for example, L. M. Harwood, C. J. Moody, Experimental Organic Chemistry, Blackwell Scientific Publications, UK 1989).

### Botanical Compositions

As noted above, the plant extracts can be included in the system of the present invention in the form of botanical compositions. In their simplest embodiment, the botanical compositions of the present invention consist of a plant extract or combination of plant extracts prepared as described above.

Alternatively, the botanical composition can be prepared by formulating the extract(s) according to standard techniques known in the art for the preparation of formulations intended for topical use (see, for example, “Remington: The Science and Practice of Pharmacy” (formerly “Remingtons Pharmaceutical Sciences”); Gennaro, A., Lippincott, Williams & Wilkins, Philadelphia, Pa. (2000)).

For example, the extracts can be combined with one or more preservatives and/or anti-oxidants to improve the stability and/or shelf life of the composition. Examples of suitable preservatives aid anti-oxidants include, but are not limited to, propylene glycol, parabens (such as isopropylparaben, isobutylparaben, methylparaben and propylparaben), diazolidinyl urea, essential oils (such as oils from caraway, cinnamon, clove, cumin, eucalyptus, lavender, lemon, rose, rosemary, sage, sandalwood and thyme), grapefruit seed extract and vitamin E oil (such as T-50 vitamin E oil), or combinations thereof.

The present invention also contemplates the formulation of the botanical compositions by mixing the extract(s) with a physiologically acceptable carrier. Excipients, binders, diluents, and other additives, such as preservatives, stabilizers, emulsifiers, buffers, colouring agents, fragrances, anti-oxidants, thickening agents, ultra-violet light stabilizers, and the like can also be included in the final composition. Other active ingredients including other plant extracts, moisturizers, vitamins and minerals and the like, can also be added. When the composition comprises more than one extract, the extracts can be formulated together to provided the botanical composition, or the extracts can be formulated independently and the respective formulations subsequently combined using a diluent or the like to provide the final botanical composition. Alternatively, each extract can be formulated independently and maintained as a separate botanical composition. As yet another alternative, the extracts(s) can be formulated independently, followed by mixing of at least two formulations to provide one botanical composition, and the other extract(s) can be maintained as separate botanical compositions.

Thus, in one embodiment of the invention, the botanical composition comprises two or more extracts that are mixed together followed by the addition of physiological carriers. In another embodiment of the invention, the botanical composition comprises two or more extracts that are formulated individually and then mixed. In still another embodiment, two or more extracts are formulated individually and kept as separate compositions.

In a specific embodiment, the system comprises one botanical composition that includes one or more plant extracts from steroidal alkaloid-containing plants, a Pilocarpus plant and a seaweed extract. In another embodiment, the system comprises one or more plant extracts from steroidal alkaloid-containing plants, an extract from a Pilocarpus plant and a seaweed extract provided as two or more botanical compositions. In another embodiment, the system comprises a first botanical composition including one or more plant extracts from steroidal alkaloid-containing plants, a second botanical composition including an extract from a Pilocarpus plant and a third botanical composition including a seaweed extract. In a further embodiment, the system of the present invention comprises two or more plant extracts from steroidal alkaloid-containing plants provided as separate botanical compositions, a botanical composition including an extract from a Pilocarpus plant and a botanical composition including a seaweed extract. In yet another embodiment, the system of the present invention comprises a botanical composition including one or more plant extracts from steroidal alkaloid-containing plants and an extract from a Pilocarpus plant, and a botanical composition including a seaweed extract.

In a further embodiment, when the system of the present invention comprises an extract from a Fucus plant, the extract from the Fucus plant is formulated as a botanical composition for oral administration.

In one embodiment of the present invention, the botanical compositions comprise between about 1% to about 25% w/w or v/v of each plant extract.

The botanical compositions according to the invention may be in solid, semi-solid or liquid form, including both aqueous and non-aqueous liquid forms, and can be provided in unit dosage form where appropriate. The compositions of the invention can be provided in a variety of conventional forms including, but not limited to, solutions, aqueous suspensions, oily suspensions, dispersible powders, dispersible granules, tablets, emulsions, hydrophobic creams, hydrophilic creams, liquid creams, lotions, ointments, waxes, gels, pastes, jellies, tinctures, liniments, sprays, aerosols, sticks, on sponges or cotton applicators, or as solutions or suspensions in an aqueous liquid, a non-aqueous liquid, an oil-in-water emulsion, or a water-in-oil liquid emulsion.

Various physiologically acceptable carriers known in the art can be used to prepare the botanical compositions of the invention. Examples of suitable carriers include, but are not limited to, hydroxypropyl cellulose, starch (corn, potato, rice, wheat), pregelatinized starch, gelatine, sucrose, acacia, alginic acid, sodium alginate, guar gum, ethyl cellulose, carboxymethylcellulose sodium, carboxymethylcellulose calcium, polyvinylpyrrolidone, methylcellulose, hydroxypropyl methylcellulose, microcrystalline cellulose, powdered cellulose, glucose, croscarmellose sodium, crospovidone, polacrilin potassium, sodium starch glycolate, tragacanth, calcium carbonate, dibasic calcium phosphate, tribasic calcium phosphate, kaolin, mannitol, talc, cellulose acetate phthalate, polyethylene phthalate, shellac, titanium dioxide, carnauba wax, microcrystalline wax, calcium stearate, magnesium stearate, stearic acid, sodium lauryl sulfate, zinc stearate, ethyl oleate, ethyl laurate, agar, calcium silicate, magnesium silicate, silicon dioxide, colloidal silicon dioxide, calcium chloride, calcium sulfate, silica gel, diethyl phthalate, mono- and di-acetylated monoglycerides, triacetin, alamic acid, aluminum monostearate, bentonite, bentonite magma, carbomer 934, carboxymethylcellulose sodium 12, carrageenan, hydroxyethyl cellulose, magnesium aluminum silicate, pectin, povidine, sodium alginate, tragacanth, xanthan gum, silicones, glycols (such as ethylene glycol, polyethylene glycol and propylene glycol), esters, glycerine, sorbitol, mannitol, alcohols (such as ethanol, propanol, isopropanol and polyvinyl alcohol), oils (such as castor oil, mineral oil, light mineral oil, peanut oil, cottonseed oil, sunflower oil, sesame oil, olive oil, corn oil and soybean oil), lipid materials, and the like.

Compositions formulated as aqueous suspensions contain the extract(s) in admixture with excipients suitable for the manufacture of aqueous suspensions. Such excipients are suspending agents, for example, sodium carboxymethylcellulose, methyl cellulose, hydropropylmethylcellulose, sodium alginate, polyvinylpyrrolidone, gum tragacanth and gum acacia: dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example hepta-decaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxy-benzoate and/or one or more colouring agents.

Compositions can be formulated as oily suspensions by suspending the extract(s) in a vegetable oil, for example, arachis oil, olive oil, sesame oil or coconut oil, or in a mineral oil such as liquid paraffin. The oily suspensions may contain a thickening agent, for example beeswax, hard paraffin or cetyl alcohol. Colouring agents may be added and the formulations may be preserved by the addition of an anti-oxidant such as ascorbic acid.

Dispersible powders and granules suitable for preparation of an aqueous suspension by the addition of water or another carrier provide the extract(s) in admixture with a dispersing or wetting agent, suspending agent and one or more preservatives. Suitable dispersing or wetting agents and suspending agents are exemplified by those described above. Additional excipients, for example, colouring agents, may also be present.

Compositions of the invention may also be in the form of oil-in-water emulsions. The oil phase may be a vegetable oil, for example, olive oil or arachis oil, or a mineral oil, for example liquid paraffin, or mixtures of these. Suitable emulsifying agents may be naturally-occurring gums, for example, gum acacia or gum tragacanth, naturally-occurring phosphatides, for example soy bean, lecithin, and esters or partial esters derived from fatty acids and hexitol, anhydrides, for example sorbitan monoleate, and condensation products of the said partial esters with ethylene oxide, for example polyoxyethylene sorbitan monoleate. The emulsions may also contain colouring agents.

In one embodiment of the present invention, the botanical compositions are formulated with one or more carriers selected from the group of: water, glycols, esters, glycerine, oils and alcohols. An exemplary cosmetic carrier known in the art comprises an aqueous alcoholic solution containing ethanol, propanol or isopropanol, together with a lower alkyl(C₁-C₄) glycol, such as ethylene glycol or propylene glycol. A thickener or gelling agent can be added. Dimethicone and other volatile silicone solvents are also useful.

The present invention also contemplates that the botanical compositions can be formulated as in shampoo and/or conditioner form. Thus, the botanical compositions can be added to shampoo formulations commonly used in the art, such as those comprising mixtures of fatty acid esters of sorbitol and sorbitol anhydrides (polysorbates), which have non-ionic properties that inhibit shedding of hair.

The botanical compositions intended for topical application can be packaged in a suitable container to suit the viscosity of the formulation and intended use. For example, a lotion or fluid cream can be packaged in a bottle or a roll-ball applicator, a capsule, a propellant-driven aerosol device or a container fitted with a pump suitable for finger operation. When the composition is a cream, paste or gel, it can simply be stored in a nondeformable bottle or squeeze container, such as a tube or a lidded jar.

### Other Optional Active Components

One or more of the botanical compositions included in the system of the present invention can comprise one or more additional active components that supplement the ability of the system to promote hair growth and/or hair health.

For example, the compositions can include one or more moisturising agents, i.e. an agent that facilitates hydration of the scalp or hair by inhibiting or preventing loss of water from the scalp/hair, that absorbs water from the atmosphere and hydrates the scalp/hair, and/or that enhances the ability of the scalp/hair to absorb water directly from the atmosphere. Suitable moisturising agents include, but are not limited to, 2-hydroxyacetic acid (glycolic acid); 2-hydroxypropanoic acid (lactic acid); 2-methyl 2-hydroxypropanoic acid; 2-hydroxybutanoic acid; phenyl 2-hydroxyacetic acid; phenyl 2-methyl 2-hydroxyacetic acid; 3-phanyl 2-hydroxyacetic acid; 2,3-dihydroxypropanoic acid; 2,3,4-trihydroxybutanoic acid; 2,3,4,5,6-pcntahydroxyhexanoic acid; 2-hydroxydodecanoic acid; 2,3,4,5-tetrahydroxypentanoic acid; 2,3,4,5,6,7-hexahydroxyheptanoic acid; diphenyl 2-hydroxyacetic acid; 4-hydroxymandelic acid; 4-chloromandelic acid; 3-hydroxybutanoic acid; 4-hydroxybutanoic acid; 2-hydroxyhexanoic acid; 5-hydroxydodecanoic acid; 12-hydroxydodecanoic acid; 10-hydroxydecanoic acid; 16 hydroxyhexadecanoic acid; 2-hydroxy-3-methylbutanoic acid; 2-hydroxy-4-methylpentanoic acid; 3-hydroxy-4-methoxymandelic acid; 4-hydroxy-3-methoxymandelic acid; 2-hydroxy-2-methylbutanoic acid; 3-(2-hydroxyphenyl) lactic acid; 3-(4-hydroxyphenyl) lactic acid; hexahydromandelic acid; 3-hydroxy-3-methylpentanoic acid; 4-hydroxydecanoic acid; 5-hydroxydecanoic acid; aleuritic acid; 2-hydroxypropanedioic acid; 2-hydroxybutanedioic acid; tannic acid; salicylic acid; erythraric acid; threaric acid; arabimric acid; ribaric acid; xylaric acid; lyxaric acid; glucaric acid; galactaric acid; mannaric acid; gularic acid; allaric acid; altraric acid; idaric acid; talaric acid; 2-hydroxy-2-methylbutanedioic acid, citric acid, isocitric acid, agaricic acid, quinic acid, glucoronic acid, glucoronolactone, galactoronic acid, galactoronolactone, uronic acids, uronolactones, ascorbic acid, dihydroascorbic acid, dihydroxytartaric acid, tropic acid, ribonolactone, glucoiolactone, galactonolaclone, gulonolactone, mannonolactone, citramalic acid; pyruvic acid, hydroxypyruvic acid, hydroxypyruvic acid phosphate and esters thereof; methyl pyruvate, ethyl pyruvate, propyl pyruvate, isopropyl pyruvate; phenyl pyruvic acid and esters thereof; methyl phenyl pyruvate, ethyl phenyl pyruvate, propyl phenyl pyruvate; formyl formic acid and esters thereof; methyl formyl formate, ethyl formyl formate, propyl formyl formate; benzoyl formic acid and esters thereof; methyl benzoyl formate, ethyl benzoyl formate and propyl benzoyl formate; 4-hydroxybenzoyl formic acid and esters thereof; 4-hydroxyphenyl pyruvic acid and esters thereof; and 2-hydroxyphenyl pyruvic acid and esters lactones or pharmaceutically acceptable salts thereof.

Other examples of moisturising agents include ceramide, borage oil (linoleic acid), tocopherol linoleate, dimethicone, glycerine, hyaluronic acid, sodium peroxylinecarbolic acid (sodium PCA), wheat protein (such as laurdimonium hydroxypropyl hydrolyzed wheat protein), hair keratin amino acids, and mixtures thereof. Sodium chloride may also be present, for example, when hair keratin amino acids are included as a moisturiser. Other moisturising agents that may be included in the compositions include primrose oil and flax seed oil The compositions may further optionally include one or more of a cysteine component, magnesium component, manganese component, selenium component, or copper component. These components are known in the art to impart beneficial effects to the hair. Compounds to aid in the repair of hair may also be added to the compositions. Examples of such compounds include selenium and silica, which may be added in relatively isolated form or in the form of various partially processed plant material or extract (such as, Horsetail-plantain or nettle root extract).

Compounds that increase circulation to the cells of the scalp can be added to the compositions, for example, thistle extract, Ginkgo Biloba extract, pepper extract (including Cayenne pepper and Red pepper), and ursolic acid.

Antimicrobial agents may also be added to the compositions. Examples of antimicrobial agents include, but are not limited to, organic solvents (such as alcohols), plant oils or extracts (such as oil of wintergreen, tea tree oil, peppermint oil, caraway oil, cinnamon oil, clove oil, cumin oil, eucalyptus oil, lavender oil, lemon oil, rose oil, rosemary oil, sage oil, sandalwood oil, thyme oil or grapefruit seed extract) and ursolic acid.

Examples of other plant extracts that can be added to the compositions include, Abrus precatorius, Sarsaparilla (Smilax officinalis), Eclipla alba (leaf), Wedelia calendula (leaf), Terminalia belerica roxb (fruit), and Terminalia Chebuto Retz.

### Testing The Plant Extracts and Botanical Compositions

The efficacy of the plant extracts and/or botanical composition(s) to be included in the system can be tested, for example, on a panel of volunteer subjects. When the system comprises a plurality of separate extracts, or a plurality of compositions, these are generally applied sequentially to the affected areas of the volunteer subjects. For example, a panel of volunteers can apply a various doses of a test extract or composition or combination of extracts/compositions over a predetermined period of time and the improvement in hair growth and/or hair health and/or scalp health in this group can be assessed at varying intervals and compared to an appropriate control or controls. Examples of appropriate controls include groups of subjects using a product known to improve hair growth and/or hair health and/or scalp health (positive control), and/or groups of subjects using a placebo treatment, or an untreated group (negative control). Such studies can also be used to monitor any side-effects and/or additional benefits of the botanical compositions under investigation by compiling reports of any positive or negative effects encountered during the course of the study in comparison to the control group(s). Optimal treatment times and quantities of the test composition to be applied can also be determined in this manner. Such studies are routine in the art and can be readily designed and conducted by a skilled technician.

If desired, the plant extracts and botanical compositions can also be submitted to standard tests, such as cytotoxicity tests, stability tests, bioavailability tests and the like, to determine their suitability for human or animal use Such tests are well known in the art and are generally conducted in accordance with government-established guidelines. In some cases the toxicity or lack of toxicity of the plant extracts that are included in the system according to the present invention, may be determined by reviewing known sources of toxicity information as known in the art For example, the toxicity of Veratrum album plant extracts, may be found in CKFRAY Ceskoslovenska Fannacie. (PNS-Ustredni Expedice a Dovoz Tisku, Kafkova 19, 160 00 Prague 6, Czechoslovakia) V. 1-1952—Volume(issue)/page/year. 10,413,1961.

### Methods of Promoting Hair Growth

The present invention further provides for a method of promoting hair growth comprising administering the system of the present invention to a subject. The extracts or composition(s) of the system of the present invention are generally administered topically by application to the area in need of treatment. The extracts/compositions can be applied between four times a day and once every two weeks, depending on the severity of the condition being treated. For example, for a bald or partially bald subject, initial application of the extracts/compositions of the system between one and four times a day for about four to six weeks and up to several months would be appropriate. The frequency of application can be decreased after this time if the desired results are obtained. For example, the present invention contemplates that a maintenance level of treatment can be followed involving application of the extracts/compositions between once a day and three times a week.

Each extract/composition of the system should be applied directly to the affected area(s) and should be left in place for a period of time of at least about 15 minutes and up to and including the time the next application is due.

When the system comprises a plurality of separate extracts/compositions, these are generally applied to the affected area in sequential steps. Accordingly, if the system comprises three extracts/compositions the method comprises, as a first step, application of a first extract/composition to the affected area. After a suitable period of time, a second extract/composition is then applied to the affected area and after a suitable period of time, a third extract/composition is applied to the affected area. A suitable period of time between applications ranges from between about 1 minute to about 30 minutes. In one embodiment, a period of time between about 5 minutes to about 10 minutes is employed between applications of the respective extracts/compositions.

In another embodiment of the present invention, the method comprises applying to the affected area a first composition comprising an extract from a Pilocarpus, a second composition comprising an extract from a steroidal alkaloid-containing plant and a third composition comprising an extract from a seaweed to the affected area. In yet another embodiment of the present invention, the method comprises applying to the affected area a first composition comprising an extract from a Pilocarpus plant, a second composition comprising an extract from a Veratrum plant and/or an extract from a Rauwolfia plant and a third composition comprising an extract from a seaweed.

In another embodiment of the present invention, the method comprises applying to the affected area a first composition comprising an extract from a Pilocarpus plant and an extract from a steroidal alkaloid-containing plant, and a second composition comprising an extract from a seaweed. In yet another embodiment of the present invention, the method comprises applying to the affected area a first composition comprising an extract from a Pilocarpus plant and an extract from a Veratrum plant, and a second composition comprising an extract from a seaweed.

In a further embodiment of the invention, when the extracts of the system are provided as separate botanical compositions, the method comprises applying the compositions in suitable relative amounts or dosage units. In an embodiment of the invention when the system comprises a first composition comprising an extract from a Pilocarpus plant, a second composition comprising one or more extracts from steroidal alkaloid-containing plants, and a third composition comprising a seaweed extract, the ratio of the amount of each composition to be applied is between about 2:2:3 and about 1:4:6 of first composition: second composition: third composition. In another embodiment, the first, second and third compositions are applied in a volume ratio of about 1:2:3. In a further embodiment, the first, second and third compositions are applied in a volume ratio of about 2:5:6.

In one embodiment of the present invention, the unit dosage for a composition included in the system is between about 0.1 ml and about 10 ml. In another embodiment of the invention, the dosage unit ranges from about 0.2 ml to about 7 ml.

In another embodiment, the dosage unit ranges from about 0.4 ml to about 5 ml. In an alternative embodiment, the dosage unit ranges from about D1 grams to about 10 grams ml. In another embodiment of the invention, the dosage unit ranges from about 0.2 grams to about 7 grams. In another embodiment, the dosage unit ranges from about 0.4 grams to about 5 grams.

### Uses

The system of the present invention can be used to promote the growth of hair in a subject in regions of the body where hair growth has ceased or diminished, or where hair growth is naturally sparse, or where thicker hair growth is desirable.

The system is suitable for use by subjects experiencing complete or partial hair loss, such as that encountered in various forms of alopecia, including alopecia androgenetica (“male pattern baldness”), alopecia cicatrista and alopecia areata. Similarly, the system can be used to promote hair growth in subjects who have experienced hair loss due to trauma, injury, chemotherapy, stress, genetic factors, hormonal changes, disease, nutritional imbalance, scalp abnormalities and the like. The system can also be used by hair transplant patients. Surgical hair transplants are now fairly commonplace, however, hair transplant grafts often fall out after about 24 weeks. Although most grafts grow back after 3-4 months, additional transplant surgery may be needed. The system of the present invention can be used by hair transplant patients to help condition the scalp, prevent or reduce hair fall out, and/or reduce the “hair shock” time. The system can also be used to reduce hair loss in a subject, as well as to promote hair growth in a subject who has naturally sparse hair growth.

In one embodiment, the system can be used to enhance or restore hair colour, for example, by restoring colour to grey or white hair, or by changing or darkening the colour of the hair or enriching the existing hair colour. In another embodiment, the system has an additional application in promoting the appearance of healthy-looking hair, for example, by increasing the thickness or lustre of hair. In a further embodiment, the system of the present invention are also useful in reducing or eliminating dandruff, and/or ameliorating itching and irritation associated with dandruff, seborrheic dermatitis and psoriasis of the scalp.

The system and methods of the present invention are suitable for use in both humans and other mammalian species. For example, the system can be applied to non-human mammals used in wool or fur production to accelerate hair growth thereby permitting, for example, greater net annual wool production or reducing the time needed to produce mature pelts. Animals used in wool or fur production include, but are not limited to, alpaca, beaver, calf, chinchilla, coyote, ermine, fisher, fitch, fox, lamb, llama, lynx, marten, mink, muskrat, nutria, opossum, otter, raccoon, Russian squirrel, sable, sheep and the like.

As indicated above, the system of the present invention is intended for topical (or external) application. The present invention, however, also contemplates that the effect of the system on hair growth can be enhanced or supplemented by internal administration of one or more of the plant extracts included in the system in a substantially diluted form, for example, as a homeopathic preparation or herbal tincture. Alternatively, one or more of the plant extracts included in the system may be administered in tablet form. For example, kelp tablets in suitable unit dosages, such as 650 mg, may be administered orally in order to enhance or supplement the effect of the system on hair growth. In one embodiment of the present invention, topical application of the system is supplemented with oral administration of a Fucus extract. In another embodiment, topical application of the system is supplemented with oral administration of a Fucus vesiculosis mother tincture. In still another embodiment, topical application of the system is supplemented with oral administration of a kelp tablet twice daily. The system can also be used in conjunction with a homeopathic preparation or herbal tincture comprising another plant extract, for example, a Sarsaparilla (Smilax officinalis) extract, which can be taken orally or applied topically. Other treatments that may be used in conjunction with the system of the present invention include exposure of the treated hair or scalp to sunlight, or increased intake of drinking water.

### Kits

The present invention additionally contemplates that the above-described system can be provided in kit form. The kit comprises the extracts or composition(s) of the system in a suitable container or containers. The present invention contemplates that the extracts or compositions of the system call be provided in a ready to use format or, alternatively, in lyophilised form and the kit can further comprise reagents suitable for the reconstitution of the lyophilised extracts. The present invention further contemplates that the extracts can be provided as solutions and the kit can contain additional components to be added to the extracts to facilitate their application to affected areas. Where appropriate, the kit may also contain mixing vessels and other instruments or containers that facilitate the reconstitution or mixing of components of the kit.

The extracts/botanical compositions can be provided in a format or container that facilitates their application to the affected area of a subject. For example, the extracts/botanical compositions can be provided as lotion or fluid cream packaged in a squeezable container, a container equipped with a nozzle or roll-ball applicator or a container fitted with a pump suitable for finger operation. When the composition is provided as a cream, paste or gel, it can be provided, for example, in a squeezable container or tube. The kit can further comprise one or more suitable implements to facilitate application of the extracts/botanical compositions.

The kit comprises sufficient amounts of the components of the system for application to the subject for a prescribed length of time, for example, for a length of time between one and twelve months. When the system comprises more than one composition and the relative amounts of each composition to be applied to the affected areas is different, the kit can provide appropriate amounts of each composition. For example, in one embodiment of the invention, the system comprises a first composition comprising an extract from a Pilocarpus plant, a second composition comprising one or more extracts from steroidal alkaloid-containing plants, and a third composition comprising a seaweed extract, and the ratio of the amount of each composition to be applied is between about 2:2:3 and about 1:4:6 of first composition: second composition: third composition. Accordingly, a kit for this system can comprise the first, second and third compositions in a similar volume ratio, for example, 60 ml, 90 ml and 120 ml, respectively.

The kit can further provide an appropriate usage regimen over a prescribed period of time for the system, for example in the form of a set of instructions, generally written instructions. The kit may further comprise one or more of the plant extracts that make up the system in a substantially diluted form suitable for oral administration, for example, as a homeopathic preparation or herbal tincture. Alternatively, where appropriate, the kit may further comprise one or more of the plant extracts of the system in tablet form for oral administration. There may also be associated with the kit a notice in the form prescribed by a governmental agency regulating the manufacture, use or sale of biological products, which notice reflects approval by the agency of manufacture, use or sale for human or animal administration.

The invention will now be described with reference to specific examples. It will be understood that the following examples are intended to describe embodiments of the invention and are not intended to limit the invention in any way.

### EXAMPLES

Examples 1 to 13 below provide exemplary compositions suitable for large-scale preparation. Suitable exemplary amounts of plant material to be included in the preparation of each composition are provided for each plant, however, these can be varied as discussed above, between about 1% to about 50% w/w for the initial extraction. While Examples 1 to 13 describe compositions that comprise a combination of extracts, the information provided in these Examples can also be used to prepare systems in accordance with the present invention in which each plant extract is formulated as a separate composition.

### Example 1

### Compositions Comprising Veratrum viride, Pilocarpus microphyllus (Maranham jaborandi), and Fucus vesiculosus

| | | Amount of Plant in Final | |
|---|---|---|---|
| | | Composition (grams) | |
| Plant | Part of Plant | Composition A | Composition B |
| Veratrum viride | Seeds | 8,000 | 8,000 |
| Maranham jaborandi | Leaves | 1,000 | 2,000 |
| Fucvs vesiculosus | Whole plant | 1,000 | 10,000 |
| Total plant material: | | 10,000 | 20,000 |

### Example 2

### Compositions Comprising Veratrum album, Pilocarpus microphyllus (Maranham jaborandi), and Fucus vesiculosus

| | | Amount of Plant in Final | |
|---|---|---|---|
| | | Composition (grams) | |
| Plant | Part of Plant | Composition A | Composition B |
| Veratrum album | Whole plant | 8,000 | 8,000 |
| Maranham jaborandi | Leaves | 1,000 | 2,000 |
| Fucvs vesiculosus | Whole plant | 1,000 | 10,000 |
| Total plant material: | | 10,000 | 20,000 |

### Example 3

### Compositions Comprising Veratrum californicum, Pilocappus microphyllus (Maranbam jaborandi), and Fucus vesiculosus

| | | Amount of Plant in Final | |
|---|---|---|---|
| | | Composition (grams) | |
| Plant | Part of Plant | Composition A | Composition B |
| Veratrum californicum | Whole plant | 8,000 | 8,000 |
| Maranham jaborandi | Leaves | 1,000 | 2,000 |
| Fucus vesiculosus | Whole plant | 1,000 | 10,000 |
| Total plant material: | | 10,000 | 20,000 |

### Example 4

### Compositions Comprising Veratrum japonicum, Pilocarpus microphyllus (Maranham jaborandi), and Fucus vesiculosus

| | | Amount of Plant in Final | |
|---|---|---|---|
| | | Composition (grams) | |
| Plant | Part of Plant | Composition A | Composition B |
| Veratrum japonicum | Whole plant | 8,000 | 8,000 |
| Maranham jaborandi | Leaves | 1,000 | 2,000 |
| Fucus vesiculosus | Whole plant | 1,000 | 10,000 |
| Total plant material: | | 10,000 | 20,000 |

### Example 5

### Compositions Comprising Veratrum nigrum, Pilocarpus microphyllus (Maranham jaborandi), and Fucus vesiculosus

| | | Amount of Plant in Final | |
|---|---|---|---|
| | | Composition (grams) | |
| Plant | Part of Plant | Composition A | Composition B |
| Veratrum nigrum | Whole plant | 8,000 | 8,000 |
| Maranham jaborandi | Leaves | 1,000 | 2,000 |
| Fucus vesiculosus | Whole plant | 1,000 | 10,000 |
| Total plant material: | | 10,000 | 20,000 |

In an exemplary preparation of Composition B, 8000 g of plant material from Veratrum nigrum are mixed with 32,000 ml of alcohol to extract the plant material, 2,000 g of plant material from jaborandi are mixed with 8,000 ml of alcohol to extract the plant material, and 10,000 g of plant material from Fucus vesiculosus are mixed with 20,000 ml alcohol to extract the plant material.

### Example 6

### Compositions Comprising Buxus sempervirens, Pilocarpus microphyllus (Maranham jaborandi), and Fucus vesiculosus

| | | Amount of Plant in Final | |
|---|---|---|---|
| | | Composition (grams) | |
| Plant | Part of Plant | Composition A | Composition B |
| Buxus sempervirens | Whole plant | 8,000 | 8,000 |
| Maranham jaborandi | Leaves | 1,000 | 2,000 |
| Fucus vesiculosus | Whole plant | 1,000 | 10,000 |
| Total plant material: | | 10,000 | 20,000 |

In an exemplary preparation of Composition B, 8000 g of plant material from Buxus sempervirens are mixed with 32,000 ml of alcohol to extract the plant material, 2,000 g of plant material from jaborandi are mixed with 8,000 ml of alcohol to extract the plant material, and 10,000 g of plant material from Fucus Vesiculosus are mixed with 20,000 ml alcohol to extract the plant material.

### Example 7

### Compositions comprising Veratrum album, Buxus sempervirens, Pilocarpus microphyllus (Maranham jaborandi), and Fucus vesiculosus

| | | Amount of Plant in Final | |
|---|---|---|---|
| | | Composition (grams) | |
| Plant | Part of Plant | Composition A | Composition B |
| Veratrum album | Whole plant | 4,000 | 4,000 |
| Buxus sempervirens | Leaves and | 4,000 | 4,000 |
| | branches | | |
| Maranham jaborandi | Leaves | 500 | 2,000 |
| Fucus vesiculosus | Whole plant | 500 | 10,000 |
| Total plant material: | | 9,000 | 20,000 |

In an exemplary preparation of Composition B. 4000 g of plant material from Veratrum album are mixed with 16,000 ml of alcohol to extract the plant material, 4000 g of plant material from Buxus sempervirens are mixed with 16,000 ml of alcohol to extract the plant material, 2,000 g of plant material from jaborandi are mixed with 8,000 ml of alcohol to extract the plant material, and 10,000 g of plant material from Fucus vesiculosus are mixed with 20,000 ml alcohol to extract the plant material.

### Example 8

### Composition Comprising Veratrum nigrum, Buxus sempervirens, Pilocarpus microphyllus (Maranham jaborandi), and Fucus vesiculosus

| | | Amount of Plant in Final |
|---|---|---|
| Plant | Part of Plant | Composition (grams) |
| Veratrum nigrum | Whole plant | 4,000 |
| Buxus sempervirens | Leaves and | 4,000 |
| | branches | |
| Maranham jaborandi | Leaves | 2,000 |
| Fucus vesiculosus | Whole plant | 10,000 |
| Total plant material: | | 20,000 |

### Example 9

### Composition Comprising Veratrum viride, Buxus sempervirens, Pilocarpus microphyllus (Maranham jaborandi), and Fucus vesiculosus

| | | Amount of Plant in Final |
|---|---|---|
| Plant | Part of Plant | Composition (grams) |
| Veratrum viride | Seed | 4,000 |
| Buxus sempervirens | Leaves and | 4,000 |
| | branches | |
| Maranham jaborandi | Leaves | 2,000 |
| Fucus vesiculosus | Whole plant | 10,000 |
| Total plant material: | | 20,000 |

### Example 10

### Composition Comprising Veratrum californicum, Buxus sempervirens, Pilocarpus microphyllus (Maranham jaborandi), and Fucus vesiculosus

| | | Amount of Plant in Final |
|---|---|---|
| Plant | Part of Plant | Composition (grams) |
| Veratrum californicum | Whole plant | 4,000 |
| Buxus sempervirens | Leaves and | 4,000 |
| | branches | |
| Maranham jaborandi | Leaves | 2,000 |
| Fucus vesiculosus | Whole plant | 10,000 |
| Total plant material: | | 20,000 |

### Example 11

### Composition Comprising Veratrum japonicum, Buxus sempervirens, Pilocarpus microphyllus (Maranham jaborandi), and Fucus vesiculosus

| | | Amount of Plant in Final |
|---|---|---|
| Plant | Part of Plant | Composition (grams) |
| Veratrum japonicum | Whole plant | 4,000 |
| Buxus sempervirens | Leaves and | 4,000 |
| | branches | |
| Maranham jaborandi | Leaves | 2,000 |
| Fucus vesiculosus | Whole plant | 10,000 |
| Total plant material: | | 20,000 |

### Example 12

### Composition Comprising Buxus sempervirens, Holarrhena, Pilocarpus microphyllus (Maranham jaborandi), and Fucus vesiculosus

| | | Amount of Plant in Final |
|---|---|---|
| Plant | Part of Plant | Composition (grams) |
| Buxus sempervirens | Leaves and | 4,000 |
| | branches | |
| Holarrhena | Bark | 4,000 |
| Maranham jaborandi | Leaves | 2,000 |
| Fitcus vesiculosus | Whole plant | 10,000 |
| Total plant material: | | 20,000 |

### Example 13

### Composition Comprising Various Veratrum species, Buxus sempervirens, Holarrhena, Pilocarpus microphyllus (Maranham jaborandi), Fucus vesiculosus, Abrus precatorius and Rauwolfia serpentina

| | | Amount of Plant in Final |
|---|---|---|
| Plant | Part of Plant | Composition (grams) |
| Buxus sempervirens | Leaves and | 5,000 |
| | branches | |
| Veratrum sabadilla | Seeds | 3,000 |
| Veratrum viride | Seeds | 3,000 |
| Veratrum album | Whole plant | 5,000 |
| Holarrhena | Bark | 2,000 |
| Maranham jaborandi | Leaves | 3,000 |
| Fucus vesiculosus | Whole plant | 3,000 |
| Abrus precatorius | Seeds | 1,000 |
| Rauwolfia serpentina | Stems and | 2,000 |
| | branches | |
| Total plant material: | | 27,000 |

### Example 14

### Preparation of Large-Scale Compositions: Method A

The following provides one exemplary method of preparing the compositions shown in Examples 1-13.

Fresh and dry branchlets, leaves, and roots from the plants are chopped and pounded to a pulp and then weighed. 90%-99% V/V ethanol or denatured ethanol was added to the pulp in a ratio of 1:3 w/w plant material:solvent and mixed thoroughly. The mixture was allowed to stand in a stoppered stainless steel tank for 40 days in a dark and cool environment. Frequent shaking is required to ensure complete extraction of the relevant components from the plant material into the ethanol. After the extraction period, the suspension was left for an additional time of between about 30 minutes to 10 days to allow the larger solid particles to settle at the bottom of the vessel and provide a cloudy solution that contains small particles of debris. The solid material was then removed by filtration through a filter press or equivalent.

Finally, the filtrate was mixed with 2% w/w propylene glycol to provide the final composition.

The above composition can be further mixed with 2,000 grams of olive oil and 10,000 grams of petroleum jelly and heated between 20° C. to 100° C. with continued mixing. The mass is allowed to cool between 1 hour to 1 month to provide a composition in the form of a cream. The cream can be transferred to and stored in tightly closed containers.

### Example 15

### Preparation of Large-Scale Compositions: Method B

The following provides another exemplary method of preparing the compositions shown in Examples 1-13.

Fresh and/or branchlets, leaves, and roots from the plants are chopped and pounded to a pulp and then weighed. The pulp is then mixed with coconut oil in a ratio between 1:1 to 1:50 w/w in a stainless steel vessel. The oil temperature should be between 10° C. to 150° C. Alternatively, the plant material may be simmered gently for between one minute and 60 minutes. The plant material is then mixed with liquid paraffin in a ratio of 2:3 w/w plant material:liquid paraffin followed by mixing with 2% w/w olive oil. The oil/pulp mixture is allowed to stand for between 7 to 10 days in dark and cool place with shaking twice daily to ensure complete extraction of the relevant components from the plant material into the oil. After the extraction period, the mixture is strained through a muslin cloth. Finally, 1 to 2% of perfume is gradually added to the oil to provide the final composition.

### Example 16

### Preparation of Large-Scale Compositions: Method C

The following provides a third exemplary method of preparing the compositions shown in Examples 1-13.

The fresh and/or dried plant materials are chopped and pounded then mixed with a ratio between 1:2 to 1:20 w/w of distilled water in a stainless steel vessel. The water temperature should be between 10° C. to 150° C. Alternatively, the plant material may be simmered gently for between one minute and 60 minutes. The plant material is then mixed with ethanol 90%-99% V/V in a ratio between 1:1 to 1:20 w/w plant material:ethanol and then mixed with 0.2 to 20% w/w propylene glycol. The mixture is allowed to stand for a time between 1 hour and 100 days in a dark and cool environment. Frequent shaking is required to ensure complete extraction of the relevant components from the plant material into the ethanol. After the extraction period, the suspension can be left for an additional time of between about 30 minutes to 10 days to allow the larger solid particles to settle at the bottom of the vessel and provide a cloudy solution that contains small particles of debris. The solid material is then removed by filtration through a filter press or equivalent.

### Example 17

### Effect of a Composition comprising Veratrum album, Pilocarpus microphyllus (Maranham jaborandi), and Fucus vesiculosus on Hair Growth and Hair and Scalp Health

Over 1000 individuals (both male and female, age ranging between 22 years to 5 years and older) in Bangladesh, India, Nepal, Singapore, Pakistan and Canada (province of Newfoundland and Labrador) topically applied about 0.5-2.0 grams (about 0.5 to 3.0 cubic cm) of the composition described in Example 2 (composition B) to the scalp two or three times per day for 3-12 months. The extracts of the composition were prepared as follows.

The Veratrum album extract was prepared by mixing 8000 grams of plant material with 40,000 ml of alcohol.

The Jaborandi extract was prepared by mixing 2000 grams of plant material with 10,000 ml of alcohol.

The Fucus vesiculosus extract was prepared by mixing 10,000 grams of plant material with 50,000 ml of alcohol.

Although the above extracts were prepared with a ratio of plant material:solvent of 1:5 and these were included in the final compositions, more concentrated forms of the extracts could also be used in which a ratio of plant material:solvent is 1:4 to 1:2.

The composition was applied by rubbing the above amount into the scalp and roots of the hair in the area(s) to be treated using the fingertips. Rubbing was continued from about one minute. Multiple doses help to ensure the homogeneity of distribution. For some subjects, exposure of the treated hair or scalp to sunlight improved results.

All subjects demonstrated new hair growth within 4-6 weeks of using the composition.

Among the test group of 1,000 subjects, 300 males subjects were at the initial stages of male pattern baldness and 300 male subject were completely bald. These subjects also took a tincture of Fucus vesiculosus (5-10 mL; prepared as per the Homeopathic Pharmacopoeia) orally before meals. All 600 of these subjects observed thickening and darkening of the very thin and weak hair within 1-3 days of using the composition. After 4-6 weeks of applying the composition, conversion of the new hair into terminal hair was observed. In some subjects, partial or general whitening of the regrown hair or vitiligo in the areas of new hair growth was observed. Vellus hairs resembling peach fuzz appeared in the thinning or bald spots in some subjects, particularly at the peripheries. The new hair became thicker and darker within 34 weeks of continued use of the composition.

Amongst the test group, 600 subjects were suffering from substantial hair loss. In these subjects, reduction of hair loss to normal or less than normal levels were reported after three to six applications.

Amongst the test group, 50 subjects who suffering from alopecia areata on the head, beard, mustache and eyebrows observed considerable hair growth and a reduction in the size of the affected areas within 4-6 weeks.

Amongst the test group, 50 female subjects suffering from hair loss due to female type alopecia observed new hair growth within 4-6 weeks. These subjects also took a tincture of Fucus vesiculosus (5-10 mL; prepared as per the Homeopathic Pharmacopoeia) orally before meals. New hair growth increased significantly as application of the composition was continued.

For those patients suffering from heredity alopecia use of the composition should be continued at least once a day or at least three times a week after restoration of hair growth.

In addition to the effects of the composition on hair growth, the following beneficial effects on the scalp and hair were reported.

Amongst the test group, 400 male and female subjects who had been suffering from dandruff observed the disappearance of the condition within 1-2 weeks of starting to use the composition.

400 male and female subjects reported improvements in hair thickness and general hair quality after five to ten applications of the composition.

Some of the subjects were suffering from hair lice and observed that this condition cleared after the third application of the composition.

In all subjects, both the newly grown and existing hair became conditioned, thicker, stronger, more attractive and lively during treatment. Restoration of original hair colour was observed within four to six weeks in almost all subjects whose hair was silver white or grey, including subjects over 60 years of age.

### Example 18

### Compositions Comprising Veratrum album, Pilocarpus microphyllus (Maranham jaborandi), Rauwolfia serpentina and Fucus vesiculosus

| | | Amount of Plant in Final | |
|---|---|---|---|
| | | Composition (grams) | |
| Plant | Part of Plant | Composition A | Composition B |
| Veratrum album | Whole plant | 8,000 | 10,000 |
| Maranham jaborandi | Leaves | 1,000 | 2,500 |
| Rauwolfia serpentina | Stem and | 1,000 | 2,500 |
| | branches | | |
| Fucus vesiculosus | Whole plant | 1,000 | 10,000 |
| Total plant material: | | 11,000 | 25,000 |

### Example 19

### Compositions Comprising Veratrum californicum, Pilocarpus microphyllus (Maranham jaborandi), Rauwolfia serpentina and Fucus vesiculosus

| | | Amount of Plant in Final | |
|---|---|---|---|
| | | Composition (grams) | |
| Plant | Part of Plant | Composition A | Composition B |
| Veratrum californicum | Whole plant | 8,000 | 10,000 |
| Maranham jaborandi | Leaves | 1,000 | 2,500 |
| Rauwolfia serpentina | Stem and | 1,000 | 2,500 |
| | branches | | |
| Fucus vesiculosus | Whole plant | 1,000 | 10,000 |
| Total plant material: | | 11,000 | 25,000 |

### Example 20

### Large Scale Preparation of Compositions: Method D

The fresh and/or dried plant material was ground to a powder of mesh size 40-80 and pounded. The powder was then mixed in a ratio between 1:2 to 1:10 (w/v) with ethanol or denatured (with isopropyl alcohol) ethyl alcohol in a stainless steel or glass vessel. For example 1 kg of plant material was mixed with 2 L of alcohol. The mixture was then allowed to stand for a time between 21 days to 100 days in the dark at a temperature of 30 to 40° C., or in an incubator. The mixture was frequently shaken to ensure complete extraction of the relevant components from the plant material into the ethanol. After the extraction period, the suspension was left for an additional time of between about 30 minutes to 10 days to allow the powder particles to settle at the bottom of the vessel and provide a cloudy solution (extract) that contains small particles of debris. The powder material was then removed by filtration through a filter press or equivalent. The powder material (plant powder) was then placed into a calcining dish and incinerated. When the plant powder turned into ash and had a light gray color, the ash was placed onto a stove top and heated it until it was pure white. When the plant powder turned white, it was allowed to cool. The ash was mixed with 2 to 20% w/w propylene glycol and placed into a tank holding the extract. The tank was sealed and the mixture allowed to stand for a time between 21 days to 100 days in the dark at a temperature of 30 to 40° C., or in an incubator, with frequent shaking to ensure complete extraction of the relevant salt material into the herbal extract. After the extraction period, the suspension was left for an additional time of between about 30 minutes to 30 days to allow the impurities suspended in the tincture to settle at the bottom of tank.

### Example 21

### Compositions Comprising Pilocarpus microphyllus (Maranham jaborandi), Veratrum album, and Fucus vesiculosus

| | | Amount of Plant in Final | |
|---|---|---|---|
| | | Composition (grams) | |
| Plant | Part of Plant | Composition A | Composition B |
| Maranham jaborandi | Leaves | 2,000 | 4,000 |
| Veratrum album | Whole plant | 8,000 | 8,000 |
| Fucus vesiculosus | Whole plant | 10,000 | 12,000 |
| Total plant material: | | 20,000 | 24,000 |

### Example 22

### Preparation of Compositions Comprising Pilocarpus microphyllus (Maranham jaborandi), Veratrum album, and Fucus vesiculosus Extracts

Extracts of Maranham jaborandi, Veratrum album and Fucus vesiculosus were prepared by Method D as described generally in Example 20 using amounts of plant material as noted below. Additional details are provided below.

Preparation of Maranham jaborandi Extract

For the Jaborandi extract, 1,000 grams of dried plant was ground to a powder and mixed with 4000 ml ethanol denatured with 7.8% isopropyl alcohol and 3.3% ethyl acetate. The mixture was mixed well and poured into an aluminium jar and allowed to stand for 30 days in the dark in a wooden box Styrofoam incubator at a temperature of 30° C. During this extraction period the jar was shaken three times daily. After the extraction period, the suspension was left for 2 days to allow the powder particles to settle at the bottom of the vessel. The extract was then decanted and stored in another aluminium jar, while the settled powder particles, or marc, was placed in a calcination dish and incinerated.

After the calcination step, the ash was mixed with 2% w/w propylene glycol and added to the aluminium jar containing the decanted extract and allowed to stand for 21 days in the dark at a temperature of 30° C. After the extraction period, the suspension was left for 5 days to allow the impurities suspended in the tincture to settle at the bottom of tank. The extract was then filtered and stored in bottles.

### Preparation of Veratrum Album Extract

For the Veratrum album extract, 8,000 grams of dried plant material was ground to a powder and mixed with 32000 ml ethanol denatured with 7.8% isopropyl alcohol and 3.3% ethyl acetate. The mixture was mixed well and poured into an aluminium jar and allowed to stand for 30 days in the dark in a wooden box Styrofoam incubator at a temperature of 30° C. During this extraction period the jar was shaken three times daily. After the extraction period, the suspension was left for 2 days to allow the powder particles to settle at the bottom of the vessel. The extract was then decanted and stored in another aluminium jar, while the settled powder particles, or mare, was placed in a calcination dish and incinerated.

After the calcination step, the ash was mixed with 2% w/w propylene glycol and added to the aluminium jar containing the decanted extract and allowed to stand for 21 days in the dark at a temperature of 30° C. After the extraction period, the suspension was left for 5 days to allow the impurities suspended in the tincture to settle at the bottom of tank. The extract was then filtered and stored in bottles.

### Preparation of Fucus Vesiculosus Extract

For the Fucus vesiculosus extract, 10,000 grams dried plant material was ground to a powder and mixed with 20,000 ml ethanol denatured with 7.8% isopropyl alcohol and 3.3% ethyl acetate The mixture was mixed well and poured into an aluminium jar and allowed to stand for 30 days in the dark in a wooden box Styrofoam incubator at a temperature of 30° C. During this extraction period the jar was shaken three times daily. After the extraction period, the suspension was left for 2 days to allow the powder particles to settle at the bottom of the vessel. The extract was then decanted and stored in another aluminium jar, while the settled powder particles, or mare, was placed in a calcination dish and incinerated.

After the calcination step, the ash was mixed with 2% w/w propylene glycol and added to the aluminium jar containing the decanted extract and allowed to stand for 21 days in the dark at a temperature of 30° C. After the extraction period, the suspension was left for 5 days to allow the impurities suspended in the tincture to settle at the bottom of tank. The extract was then filtered and stored in bottles.

### Example 23

### Effect of Separately Applied Veratrum album, Pilocarpus microphyllus (Maranham jaborandi), and Fucus vesiculosus Compositions on Hair Growth and on Hair and Scalp Health

Ten volunteer patients consisting of nine men and one woman exhibiting pattern baldness were enrolled in the testing in Canada. These patients ranged in age between 30 years to 55 years All participants were in apparent good health and none had been previously involved in any studies or treatments of this type. Of the 10 volunteers, 4 subjects had male pattern baldness for 10-15 years, 2 male subjects and the 1 female subject had vertex and frontal baldness for 5 years, and 2 male subjects were completely bald with smooth patches of glossy scalp skin, and 1 male subject had thin frontal hair which had developed into completely smooth patches giving a frontal bald area of 7×5 cm. This subject had previously taken orally Medicago sativa (alfalfa) herbal tablets.

The patients used a treatment system comprising the three compositions described in Example 22. The compositions were applied two or three times daily, for 3 months. The method of application was as follows. Each composition was applied by rubbing the volumes specified below into the smooth patches of the scalp and roots of the hair in the area(s) to be treated using the fingertips. Rubbing was continued for about one minute. If necessary, multiple applications could be used to ensure a homogeneous distribution of the applied composition. The composition comprising the Jaborandi extract was applied to the affected area first in a volume of about 30-60 drops (about 1 to 4 ml).

After 5-10 minutes, the composition comprising the Veratrum album extract was applied in a volume of 60-120 drops (about 4 to 8 ml). A mild burning sensation and sneezing was observed by local application of this second extract. After 5-10 minutes, 90-180 drops (about 8 to 16 ml) of the composition comprising the Fucus vesiculosus extract was applied.

Without being limited to any particular mechanism, it is believed that the first treatment step aids in removing sebum from the follicle entrance and open the pores of the skin of the scalp (sebum is a secretion composed of natural and non polar lipids and may interfere with the accessibility of the composition to the follicle); the second treatment, which was allowed to penetrate the hair follicle, aids in stimulating the hair nerves and stem cells around the bulge region of the follicles and the third treatment aids in providing nutrition to the new grown fine hair and decreasing skin thickness.

All subjects also took a tincture of Fucus vesiculosus (5-10 ml; prepared as per the American Homeopathic Pharmacopeia) orally twice before meals. For some subjects exposure of the treated hair or scalp to sunlight and drinking plain water improved results.

All subjects were individually and carefully instructed on the proper scalp application of the compositions, oral consumption of the supplemental tincture and the botanical compositions.

All subjects demonstrated new hair growth 2-4 weeks of using the composition.

The regrowth of hair was first noticed in some subjects as early as two weeks into the treatment. By the third week a substantial number of men and women demonstrated moderate regrowth, both fine vellus hairs and darker pigmented intermediate and terminal hairs were observed. There was some partial and general whitening of the hair that regrew after treatment. Some of the fine vellus hairs visible by magnifying glass to the naked eye did not reproduce in the photographs of the scalp due to the relative lack of sensitivity of the latter.

Photographs and initial counts were taken prior to the treatment using a normal 2× magnifying lens. Independent counts were made by two individuals. From the photographs taken during the course of study, a pattern of regrowth was observed in those participants who demonstrated a significant increase in hair counts over three months. The first countable hairs were seen after 4-6 weeks treatment and were first detected at the hairline in the lateral frontal region and on the crown or vertex of the head including outer third of the eyebrows. Following further treatment, hair growth was then observed ii the frontal temporal region of the scalp after 6-12 weeks of treatment.

Some of the subjects who were suffering from dandruff prior to treatment observed the disappearance of the condition within 1-2 weeks of the start of treatment. In all subjects, both the newly grown and existing hair become conditioned, thicker, darker, stronger, more attractive and/or lively during treatment. Some of the subjects who were suffering from scalp problems such as psoriasis prior to treatment observed this condition cleared after third application of the treatment.

In the first month, the average hair count in responders was 30-60 hairs per square inch. The number of years the patient had been affected with baldness or thinning hair did not appear to have any correlation with initial results. All the users said they were satisfied with their frontal hairline and were satisfied with the top of their head.

The results indicated that the treatment system appears to be as effective as, or more effective than, other natural and synthetic chemical products in terms of hair regrowth. In addition, the plant extracts included in the composition have been individually tested for toxicity in the past and it is unlikely, therefore, that the treatment system has any significant side effects.

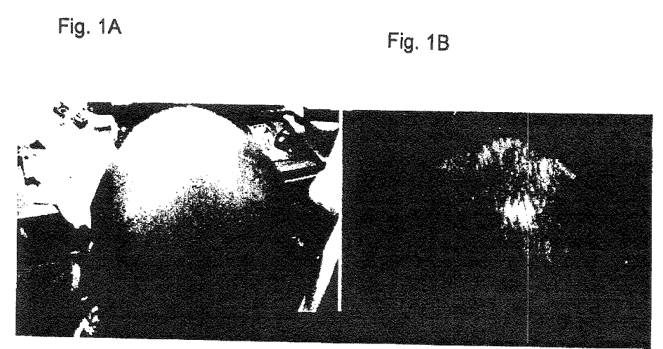

FIG. 1A depicts the affected area of one patient prior to treatment. FIG. 1B depicts the affected area of the same patient after treatment with the composition according to Example 17 (in which extracts are combined), and with the composition as described in this Example (where extracts are sequentially applied).

### Example 24

### Formulation and Application of Botanical Compositions

Exemplary formulations of botanical compositions that can be included in the described treatment system according to the present invention are shown below.

### Composition 1:

Plant: PILOCARPUS (Common name: Jaborandi)
Starting volume for extract preparation; 100 ml
Final volume of extract prepared from starting volume: 66 ml

### Source of Plant Materials: Garden or Land

### Plant Part Leaf

### Amounts Used in Extraction:

| | | |
|---|---|---|
| Dry leaf (containing moisture 10%) | 25 | grams |
| Denatured alcohol | 100 | ml |
| i.e. Percentage of dry herbs | 25% | |

### Amounts in Final Composition:

| Percentage of alcohol | 94.25% (measured using |
|---|---|
| | an alcohol meter) |
| Percentage of Pilocarpus extract | 2.75% |
| Percentage of propylene glycol | 2% |
| Percentage of nitric acid | 1% |

### Recommended Use:

| | |
|---|---|
| Duration of use | 3 to 6 Months (or as per |
| | ratio of hair falling) |
| Route of Administration | Topically |
| Amount | 2 ml. |
| Dosage Unit | 2 ml. |
| Frequency | Twice or thrice daily |

### Composition 2:

Plant: VERATRUM ALBUM (Common name: White hellebore)
Starting volume for extract preparation: 100 ml
Final volume of extract prepared from starting volume: 66 ml

### Source of Plant Materials: Forest

### Plant Part Branch

### Amounts Used in Extraction:

| | | |
|---|---|---|
| Dry branch (containing moisture 10%) | 25 | grams |
| Denatured alcohol | 100 | ml |
| i.e. Percentage of dry herbs | 25% | |

### Amounts in Final Composition:

| Percentage of alcohol | 95% (measured using |
|---|---|
| | an alcohol meter) |
| Percentage of Veratrum album extract | 2% |
| Percentage of propylene glycol | 2% |
| Percentage of nitric acid | 1% |

### Recommended Use.

| | |
|---|---|
| Duration of use | 3 to 6 Months (or as per |
| | ratio of hair falling) |
| Route of Administration | Topically |
| Amount | 5 ml |
| Dosage Unit | 5 ml |
| Frequency | Twice or thrice daily |

Plant: FUCUS VESICULOSUS (Common names: Bladderwrack, Sea weed, Kelp)
Starting volume for extract preparation: 100 ml
Final volume of extract prepared from starting volume: 66 ml

### Source of Plant Materials: Ocean

Plant Part Whole plant

### Amounts Used in Extraction:

| | | |
|---|---|---|
| Dry plant (containing moisture 15%) | 50 | grams |
| Denatured alcohol | 100 | ml |
| i.e. Percentage of dry herbs | 50% | |

### Amounts in Final Composition:

| Percentage of alcohol | 95% (measured using |
|---|---|
| | an alcohol meter) |
| Percentage of Fucus vesiculosus extract | 2% |
| Percentage of propylene glycol | 2% |
| Percentage of nitric acid | 1% |

### Recommended Use:

| | |
|---|---|
| Duration of use | 3 to 6 Months (or as per |
| | ratio of hair falling) |
| Route of Administration | Topically |
| Amount | 6 ml |
| Dosage Unit | 6 ml |
| Frequency | Twice or thrice daily |

### Example 25

### Exemplary Method for Production of Flair Lotion

The following describes an exemplary method used to produce a hair lotion that can be included in the system according to the present invention.

An extraction process to isolate the active ingredients from plants and herbs is described as follows.

All herbs and plants used to prepare the lotion should be free of any chemicals or preservatives, for example, pharmaceutical grade raw herbs. The quality and identification of all herbs may be checked by botanical and pharmacological experts using, for example visual inspection or laboratory analysis using sophisticated scientific equipment available. Thin layer chromatography or other techniques can be used to identify the species and the presence of active compounds, if desired, for analysis of chemical substances.

As botanical substitution is an increasing problem, the authenticity of the botanical species may be checked, for example, by high performance thin layer chromatography (HPTLC) and compared to a certified standard.

After the identity of the raw materials has been established, they may be further tested by HPLC to ensure that the material contains adequate levels of active constituents or marker compounds. It can be useful to compare the HPLC graph of the raw material with the certified standard. The analysis can be further enhanced by applying the photodiode array detector (PDA) to the HPLC analysis. The PDA produces a three-dimensional graph which assists examining purity of each of the peaks.

Ultraviolet and visible spectroscopy (UV/VIS) is the measurement of the wavelength and intensity of absorption of ultraviolet and visible light by a sample and can be used for quantitative measurements. UV/VIS can be used as an in-process test method, that is to check the effectiveness of an extraction in progress. Results from UV/VIS testing can be backed up by HPLC to verify the result. A UV/VIS test of a milk thistle extract may, for example, give a reading of 30% silymarin, whereas the more accurate reading from HPLC may only be 20%.

The extraction process can be chosen to minimize decreasing the activity of the bioactive compounds.

### Basic Process

The raw herbs are checked for herbicides, pesticides, heavy metal content, and other harmful substances. When the system comprises botanical compositions that are formulated individually and kept as separate compositions, the separate compositions should be made of uniform strength. When plants are collected from their natural habitat, they are said to be “wild-crafted”. When they are grown utilizing commercial farming techniques, they are said to be “cultivated”. A collection of plants from cultivated sources ensures that the plant collected is the one that is desired. When a herb is wild-crafted, there is a much greater chance that the wrong herb will be picked. The use of analytical techniques can be employed to guarantee that the plant collected is the one desired.

Garbling refers to the separation of that portion of the plant to be used from other parts of the plant, dirt, and other extraneous matter. This step is often performed during the collection process and may be performed by machines.

Herbs and plants vary considerably in their moisture content depending on the different conditions under which they are grown, collected and preserved. After harvesting, most herbs have a moisture content of 60-90%, and may need to be dried prior to storage to minimize breakdown of important compounds, or microorganism contamination. The majority of herbs require relatively mild conditions for drying Commercially, most plants are dried within a temperature range of 100-140° F. During drying, the plant material must not be damaged, or suffer losses, that would prevent from conforming to accepted composition standards. With proper drying, the herb moisture content will be reduced to less than 14%.

Grinding or mincing an herb means mechanically breaking down either leaves, root seeds, or other parts of a plant into very small units ranging from larger course fragments to fine powder. Grinding is employed in the production of crude herb products as well as in the initial phases of extracts.

Often the material has to be pre-chopped, or minced, before feeding it into a grinder. In the process of grinding, a number of machines can be used, but the most wide used is the hammer mill. These machines are simple in design. The hammer arranged radially, follow the rotation of the shaft to which they are attached, breaking up the material that is fed into the machine from above. On the walls of the chamber is a grid, which determines the size of the material that is passed through it. Other types of grinders include knife mills and tooth mills.

The plants and herbs are extracted by maceration in solvent at a predetermined temperature in incubator.

After extraction of the herb, the resulting solutions can be concentrated into fluid extracts or solid extracts. For large scale preparation, techniques and machines, such as thin layer evaporators, known in the art can be used that ensure the extracted plant components are not damaged. These machines work by evaporating the solvent; thus leaving the plant compounds behind. The solvent vapors pass into a condenser whereby they return to a liquid state, and can then be reused. The result is separation of the extracted materials from the solvent such that the final product is a substantially pure plant extract.

The disclosure of all patents, publications, including published patent applications, and database entries referenced in this specification are specifically incorporated by reference in their entirety to the same extent as if each such individual patent, publication, and database entry were specifically and individually indicated to be incorporated by reference.

The embodiments of the invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

1. A system for promoting hair growth, said system comprising one or more of: an extract from a Veratrum plant, an extract from a Buxus plant, an extract from a Holarrhena plant, an extract from a Solanum plant, and an extract from a Rauwolfia plant.

2. The system according to claim 1, further comprising one or more extracts from Pilocarpus plants.

3. The system according to claim 1, further comprising one or more seaweed extracts.

4. The system according to claim 1, wherein said system comprises one or more extracts from Veratrum plants, one or more extracts from Buxus plants, one or more extracts from Holarrhena plants, and one or more extracts from Rauwolfia plants.

5. The system according to claim 1, wherein said system comprises one or more extracts from Veratrum plants and one or more extracts from Buxus plants.

6. The system according to claim 1, wherein said system comprises one or more extracts from Buxus plants and one or more extracts from Holarrhena plants

7. The system according to claim 1, wherein said system comprises one or more extracts from Veratrum plants and one or more extracts from Rauwolfia plants.

8. The system according to claim 1, wherein said system comprises one or more extracts from Veratrum plants.

9. The system according to claim 1, wherein said system comprises one or more extracts from Buxus plants.

10. The system according to claim 3, wherein said seaweed is a Fucus seaweed.

11. The system according to claim 3, wherein said seaweed is Fucus vesiculosus.

12. The system according to claim 1, wherein said Veratrum plants are selected from Veratrum album, Veratrum californicum, Veratrum japonicum, Veratrum nigrum and Veratrum viride.

13. The system according to claim 1, wherein said Buxus plant is Buxus sempervirens.

14. The system according to claim 1, wherein said Holarrhena plant is Holarrhena dysenterica.

15. The system according to claim 1, wherein said Rauwolfia plant is Rauwolfia serpentina.

16. A method of promoting hair growth in a subject comprising topically administering to said subject an effective amount of each extract of the system according to claim 1.

17. A method of reducing hair loss in a subject comprising topically administering to said subject an effective amount of each extract of the system according to claim 1.

18. A method of enhancing or restoring hair colour, increasing the thickness of hair, improving the general appearance of hair, or reducing or eliminating dandruff, or a combination thereof, in a subject comprising topically administering to said subject an effective amount of each extract of the system according to claim 1.

19. A system for promoting hair growth, said system comprising an extract from a Pilocarpus plant, a seaweed extract, and one or more of: an extract from a Veratrum plant, an extract from a Buxus plant, an extract from a Holarrhena plant, an extract from a Solanum plant, and an extract from a Rauwolfia plant.

20. The system according to claim 19, wherein said system comprises an extract from a Pilocarpus plant, a seaweed extract, and one or more extracts from Veratrum plants, one or more extracts from Buxus plants, one or more extracts from Holarrhena plants, and one or more extracts from Rauwolfia plants.

21. The system according to claim 19, wherein said system comprises an extract from a Pilocarpus plant, a seaweed extract, and one or more extracts from Veratrum plants and one or more extracts from Buxus plants.

22. The system according to claim 19, wherein said system comprises an extract from a Pilocarpus plant, a seaweed extract, and one or more extracts from Buxus plants and one or more extracts from Holarrhena plants.

23. The system according to claim 19, wherein said system comprises an extract from a Pilocarpus plant, a seaweed extract, and one or more extracts from Veratrum plants and one or more extracts from Rauwolfia plants.

24. The system according to claim 19, wherein said system comprises an extract from a Pilocarpus plant, a seaweed extract, and one or more extracts from Veratrum plants.

25. The system according to claim 19, wherein said system comprises an extract from a Pilocarpus plant, a seaweed extract, and one or more extracts from Buxus plants.

26. The system according to claim 19, wherein said seaweed extract is a Fucus seaweed extract.

27. The system according to claim 19, wherein said seaweed is a Fucus vesiculosus extract.

28. The system according to claim 19, wherein said Veratrum plants are selected from Veratrum album, Veratrum californicum, Veratrum japonicum, Veratrum nigrum and Veratrum viride.

29. The system according to claim 19, wherein said Buxus plant is Buxus sempervirens.

30. The system according to claim 19, wherein said Holarrhena plant is Holarrhena dysenterica.

31. The system according to claim 19, wherein said Rauwolfia plant is Rauwolfia serpentina.

32. The system according to claim 19, wherein said system comprises a single composition containing said extract from a Pilocarpus plant, said seaweed extract, and said one or more of: an extract from a Veratrum plant, an extract from a Buxus plant, an extract from a Holarrhena plant, an extract from a Solanum plant, and an extract from a Rauwolfia plant.

33. The system according to claim 19, wherein said system comprises a first composition comprising said extract from a Pilocarpus plant, a second composition comprising said seaweed extract, and a third composition comprising said one or more of: an extract from a Veratrum plant, an extract from a Buxus plant, an extract from a Holarrhena plant, an extract from a Solanum plant, and an extract from a Rauwolfia plant.

34. A method for promoting hair growth in a subject comprising the step of topically applying to the area where promotion of hair growth is desired an effective amount of an extract from a Pilocarpus plant, an effective amount of a seaweed extract, and an effective amount of one or more of: an extract from a Veratrum plant, an extract from a Buxus plant, an extract from a Holarrhena plant, an extract from a Solanum plant, and an extract from a Rauwolfia plant.

35. The method according to claim 34, wherein said step of topically applying comprises topically applying a single composition comprising an effective amount of an extract from a Pilocarpus plant, said effective amount of a seaweed extract, and said an effective amount of one or more of: an extract from a Veratrum plant, an extract from a Buxus plant, an extract from a Holarrhena plant, an extract from a Solanum plant, and an extract from a Rauwolfia plant.

36. The method according to claim 34, wherein said step of topically applying comprises topically applying a first composition comprising said effective amount of an extract from a Pilocarpus plant, a second composition comprising said effective amount of one or more of: an extract from a Veratrum plant, an extract from a Buxus plant, an extract from a Holarrhena plant, an extract from a Solanum plant, and an extract from a Rauwolfia plant, and a third composition comprising said effective amount of a seaweed extract.

37. The method according to claim 36, wherein said step of topically applying comprises sequentially:
(a) topically applying said first composition,
(b) topically applying said second composition; and
(c) topically applying said third composition.

38. The method according to claim 34, wherein said step of topically applying comprises topically applying an extract from a Pilocarpus plant, a seaweed extract, one or more extracts from a Veratrum plant, one or more extracts from a Buxus plant, one or more extracts from a Holarrhena plant, and one or more extracts from a Rauwolfia plant.

39. The method according to claim 34, wherein said step of topically applying comprises topically applying an extract from a Pilocarpus plant, a seaweed extract, one or more extracts from Veratrum plants and one or more extracts from Buxus plants.

40. The method according to claim 34, wherein said step of topically applying comprises topically applying an extract from a Pilocarpus plant, a seaweed extract, one or more extracts from Buxus plants and one or more extracts from Holarrhena plants.

41. The method according to claim 34, wherein said step of topically applying comprises topically applying an extract from a Pilocarpus plant, a seaweed extract, one or more extracts from Veratrum plants and one or more extracts from Rauwolfia plants.

42. The method according to claim 34, wherein said step of topically applying comprises topically applying an extract from a Pilocarpus plant, a seaweed extract, and one or more extracts from Veratrum plants.

43. The method according to claim 34, wherein said step of topically applying comprises topically applying an extract from a Pilocarpus plant, a seaweed extract, and one or more extracts from Buxus plants.

44. The method according to claim 34, wherein said seaweed extract is a Fucus seaweed extract

45. The method according to claim 34, wherein said seaweed is a Fucus vesiculosus extract.

46. The method according to claim 34, wherein said Veratrum plants are selected from Veratrum album, Veratrum californicum, Veratrum japonicum, Veratrum nigrum and Veratrum viride.

47. The method according to claim 34, wherein said Buxus plant is Buxus sempervirens.

48. The method according to claim 34, wherein said Holarrhena plant is Holarrhena dysenterica.

49. The method according to claim 34, wherein said Rauwolfia plant is Rauwolfia serpentina.

50. The method according to claim 34, wherein said method further comprises orally administering to said subject an extract from a Pilocarpus plant, a seaweed, a Veratrum plant, a Buxus plant, a Holarrhena plant, or a Rauwolfia plant.

51. The method according to claim 34, wherein said method further comprises orally administering to said subject a seaweed extract.

52. A kit for promoting hair growth comprising the system according to claim 1 and optionally instructions for use.

53. A kit for promoting hair growth comprising the system according to claim 19 and optionally instructions for use.
